(19) United States
(12) Patent Application Publication
(10) Pub. No.: US 2026/0283952 A1
(43) Pub. Date: Sep. 24, 2026

(54) Cartridge for Housing a Production of a Therapeutic Microfoam, Automated Device, Method for Producing a Therapeutic Microfoam, and Microfoam

(71) Applicant: SANTA ISABEL INTERNATIONAL FOUNDATION, Panama City (PA)

(72) Inventors: Bernhard Friedrich BENDER, HERZOGENRATH (DE); André COENEN, Eschweiler (DE); Erol MEYDA, Aache (DE); Marcel KALTENBERG, Eschweiler (DE); Thomas Stefan BRONDER, Eschweiler (DE); Juan Ramón CABRERA GARCIA-OLMEDO, Granada (ES)

(21) Appl. No.: 19/484,347

(22) PCT Filed: May 15, 2024
(86) PCT No.: PCT/EP2024/063454

§ 371 Date: Nov. 13, 2025

(30) Foreign Application Priority Data
May 15, 2023 (DE) 10 2023 112 792.6

(51) Int. Cl. A61K 9/12; A61K 31/08; B01F 23/235; B01F 23/237; B01F 23/2373; B01F 27/906; B01F 35/00; B01F 35/11; B01F 35/22; B01F 35/221; B01F 35/60; B01F 35/75; B01F 101/22

(52) U.S. Cl. CPC A61K 9/122; A61K 31/08; B01F 23/235; B01F 23/2373; B01F 23/237612; B01F 23/23762; B01F 27/906; B01F 35/11; B01F 35/187; B01F 35/2207; B01F 35/221422; B01F 35/602; B01F 35/75425; B01F 35/75471; B01F 2101/22

(57) ABSTRACT

The present invention relates to a reaction vessel (160) for producing a microfoam (10), to a cartridge (100) comprising said reaction vessel and to an automated device (1) receiving said cartridge for automatically producing a microfoam, preferably a medical microfoam, particularly a sclerosant foam for use in sclerotherapy. Furthermore, the invention relates to a method for automatically producing a microfoam, preferably a medical microfoam, particularly a sclerosant foam for use in sclerotherapy as well as a microfoam obtainable by the inventive method and a computer program for use with the automated device. The therapeutic microfoam which is obtained with the invention concept device is technically superior to all microfoams of the prior art, and the foam quality is highly reproducable.

The present invention relates to a cartridge for housing a production of a therapeutic microfoam, to an automated device receiving said cartridge for automatically producing a microfoam, preferably a medical microfoam, particularly a sclerosant foam for use in sclerotherapy. Furthermore, the invention relates to a method for automatically producing a microfoam, preferably a medical microfoam, particularly a sclerosant foam for use in sclerotherapy as well as a microfoam obtainable by the inventive method and a computer program for use with the automated device.

In general, a medical foam for the use in sclerotherapy is already known in the prior art. However, a complex mixing process is usually necessary to obtain the medical foam with the desired properties. Particularly, a small and uniform bubble size may be desired which provides a high degree of stability.

Factors that may be posing difficulties in the current state of the art may be caused by a lack of stability that can be a consequence of bubbles of the foam that are either too big or of a very heterogeneous size.

WO 2015/185554 A2 to Roche Rebollo and Puig Domenech with priority of 2014 discloses a container for the production of a foamed sclerosant composition, kits and systems including such a container, and methods for preparing a foamed sclerosant composition using such containers.

WO 2017/085209 A1 to Roche Rebollo with priority of 2015 relates to a container for the production of a foamed sclerosant composition, to kits and systems including such a container, to methods for preparing a foamed sclerosant composition using such containers, and further to foamed sclerosant compositions obtainable by such methods.

WO 2020/038928 A1 to Vascular Barcelona Devices with priority of 2018 discloses methods, devices, systems and kits for preparing compositions for care and repair of varicose veins.

US 2014/0047985 A1 to Electrical & Electronics Ltd. with priority of 2012 discloses an appliance for preparing a frothed or mixed beverage, with a stirrer including magnetic elements so that it can be driven magnetically by a motor without any physical linkage.

U.S. Pat. No. 8,876,618 B2 to Nestec S. A. discloses an appliance for frothing a milk-based liquid, which is a food appliance and has little or no connection to the present invention.

GB 860,883 A to Holmes discloses a construction site shearing device for producing a concrete foam, which is a heavy construction appliance and has little or no connection to the present invention.

Physician-compounded foams (PCFs) have been introduced in vein treatment with the aim of increasing efficacy and treating larger varicose veins relative to liquid sclerosants. However, foams are not all the same and, in fact, they can be dramatically different from each other. The performance of foams is highly dependent on their physical characteristics such as gas composition, the different absorption rates of nitrogen and carbon dioxide in the bloodstream, and bubble size.

PCFs offer several advantages over traditional liquid sclerosants. When injected into a vein, a cohesive foam displaces the blood (rather than mixing with it), creating better contact with the vein wall. Foam treatment offers the possibility of using lower sclerosant concentrations. This, in turn, increases the safety of foam treatment as shown in clinical trials. Furthermore, foam is echogenic, which improves visibility and treatment accuracy. Also, foam treatment can be performed in an outpatient setting without need for sedation or tumescent anesthesia.

Foam treatment also presents challenges. Room air (RA) forms stable foam, but because of the nitrogen contained therein, this foam does not dissolve efficiently in blood. As a result, nitrogen bubbles may persist and can cause serious complications adverse such as neurological events or the appearance of large gas bubbles in the cardiovascular system such as in the right heart chamber, in the patent foramen ovale (PFO) or other right-to-left shunts. These neurological events have been attributed to nitrogen/air as gas used for the foam generation. Foams containing a mixture of carbon dioxide and oxygen show fewer side effects but have the disadvantage that the increased solubility results in foams that coarsen rapidly, leading to drastically reduced stability. Furthermore, they tend to generate large gas bubbles that may be potentially problematic in the circulation.

Despite the extensive development in this area, there is still a need for improvements of the production of sclerosant foam.

The present invention is therefore based on the objective of providing an enrichment, improvement or alternative to the prior art.

### SUMMARY OF THE INVENTION

The objective is solved by a cartridge of patent claim 1, by an automated device of patent claim 77, by a method of claim 95, and by a microfoam of claim 101, as well es by a reaction vessel of feature combinataion 1, a cartridge with the features of feature combination 16, an automated device with the features of feature combination 25, a method with the features of feature combination 36 as well as a microfoam with the features of feature combination 38 and the computer according to feature combination 40.

Further features and details of the invention are disclosed in the respective dependent claims, in the respective dependent feature combinations, in the description and in the drawing.

Features and details described in connection with the inventive apparatus also apply to the inventive system, the inventive method, the inventive microfoam as well as the inventive computer program, and vice versa in each case, so that with respect to the disclosure concerning the individual aspects of the invention reference can always be made mutually.

Also, features and details described in connection with the patent claims also apply to the feature combinations, and vice versa in each case, so that with respect to the disclosure concerning the individual aspects of the invention reference can always be made mutually.

Furthermore, features and details described in connection with the patent claims with the choice of words used by the patent claims also apply to the feature combinations, and vice versa in each case, so that with respect to the disclosure concerning the individual aspects of the invention reference can always be made mutually, and the choice of denomination word is freely interchangeable. E.g., where the feature combinations and the description speak of a reaction vessel, while the patent claims speak of a reaction unit, these expressions are freely interchangeable. The same applies for all further parallel uses of different denominations.

In a first aspect of the present invention, according to the advantageous feature combinations, there is provided a reaction vessel for producing a microfoam that contains a reaction unit body defining a foaming chamber and containing:
a. a rotatable foaming element arranged in the foaming chamber for performing a foaming process to produce the microfoam from a liquid foaming composition,
b. a positioning member on the bottom of the foaming chamber that holds the foaming element to be rotatable around a predefined axis during the foaming process,
c. a first outlet channel provided adjacent to or at the bottom part of the foaming chamber that is in fluid communication with the said foaming chamber for removing the microfoam from the foaming chamber.

The apparatus according to the invention may have the advantage that a microfoam can be produced that has a high degree of stability, which is especially beneficial for the use of the microfoam in diagnostic or therapeutic procedures. Due to the high degree of stability the physician has enough time to retrieve the microfoam from the foaming chamber and administer it to the subject in need thereof.

The microfoam of the invention is made from small bubbles and exhibits a narrow bubble size distribution which offers high stability and cohesion enabling a widespread use in the treatment and diagnosis of patients in need thereof.

With the reaction vessel, the cartridge and the automated device of the invention, the present invention provides a basic technological platform of the highly controlled generation of stable microfoam which can be used in a broad range of applications, encompassing diagnosis, therapy or cosmetic uses.

As the inventors found out, the positioning member allows a highly precise location of the rotatable foaming element even in the rotation modus.

In a preferred embodiment, the rotatable foaming element is magnetically driven by an actuator which does not enter the foaming chamber but is located outside by e.g., a magnetic stirrer. A magnetic drive has the disadvantage that the (unguided) rotatable foaming element can be set into a tumbling motion, especially in phases of acceleration or deceleration. The positioning member prevents any tumbling motion.

In order to generate a high-quality microfoam, it has been found that the rotatable foaming element has to maintain a preselected distance to the bottom and the walls of the foaming chamber during the foaming process. This is achieved by the claimed positioning member.

The use of a rotatable foaming element which can be driven by an actuator which does not enter the foaming chamber and the provision of a reaction vessel with defined inlet and outlet channels allows the preparation of sterile microfoam as required for diagnostic or therapeutic purposes.

Hence, the claimed reaction vessel enables the automated and controlled production of a high quality microfoam in a reproducible manner.

The reaction vessel can be used with the foaming agents and aqueous diluents which are established in the prior art and can be thus used for a broad range of foaming applications.

The claimed reaction vessel allows a foam generation for which relevant parameters such as gas mixture ratios, concentrations of the foaming agent and the aqueous diluent, volume of microfoam to be generated, and the rotation parameters can be easily adapted to the therapeutic requirements.

### More in Detail:

The rotation of the rotatable foaming element leads to a thorough mixing of the contents of the foaming chamber, namely the diluted foaming agent and the gas or gas mixture and thereby creates a microfoam.

The continuous thorough mixing is supported by a safe positioning of the rotatable foaming element which is a result of the positioning member.

Preferably, the positioning member is an axis, holding the rotatable foaming element at the preselected position within the foaming chamber.

According to one option, the first outlet channel is provided adjacent to or at the bottom part of the foaming chamber that is in fluid communication with the said foaming chamber for removing the microfoam from the foaming chamber. It has been shown that the best microfoam is given in the lower part of the foaming chamber and a retrieval of the microfoam adjacent to or at the bottom of the chamber gives the best results.

“Adjacent to” is to be understood as being within a distance of max. 10 mm from the inner bottom surface.

The foaming chamber may have a cylindrical shape or an upwardly tapering conical, frustoconical, hemispherical or dome-like shape so that the predominant capacity is given in the lower part of the foaming chamber in the area of foam generation.

In a preferred embodiment, the foaming chamber has a flat bottom connected preferably with rounded edges to the sidewall of the foaming chamber. The rounded edges have advantages with regard to purification issues and later cleaning.

The reaction unit body can be made from metal or preferably from synthetic material. It can be made from any polymeric material available in the art. Preferable polymers include polyacrylics, polyamides, polycarbonates, polyesters, polyimides, vinyl polymers, and halogenated vinyl polymers, such as polytetrafluoroethylenes.

In a preferred embodiment the reaction unit body is made from a biocompatible material according to ISO 10993.

Expediently, the foaming chamber has a smooth inner surface to reduce adhesion of the foaming components. This property can be determined as surface roughness according to ISO 25178 whereby the surface preferably has a real roughness value Sa of less than 1 mm, preferably less than 0.1 mm, more preferably less than 0.01 mm, even more preferably less than 0.005 mm, and specifically less than 0.001 mm.

In a preferred embodiment, the reaction vessel is characterized by a reaction unit body defining the foaming chamber, whereby said reaction unit body further has one or more of the following characteristics, and preferably all of the following characteristics:
a. a first inlet channel that is in fluid communication with the said foaming chamber for supplying a gas or gas mixture to the foaming chamber, whereby the first inlet channel is preferably provided at the top part of the foaming chamber,
b1. a second inlet channel that is in fluid communication with the said foaming chamber for supplying a mixture comprising a foaming agent and an aqueous diluent to the foaming chamber, whereby the second inlet channel is preferably provided adjacent to or at the bottom part of the foaming chamber, or
b2. a second inlet channel that is in fluid communication with the said foaming chamber for supplying an aqueous diluent to the foaming chamber, whereby the second inlet channel is preferably provided at the top part of the foaming chamber, provided together with a third inlet channel that is in fluid communication with the said foaming chamber for supplying a foaming agent to the foaming chamber, whereby the third inlet channel is preferably provided adjacent to or at the bottom part of the foaming chamber.

The first inlet channel allows the supply of a gas or a gas mixture which can be any gas capable of producing a foam. The skilled person will select the appropriate gas or gas mixture based on the specific foam application.

The gas or a gas mixture is preferably a physiological gas such as CO₂ and O₂ or a mixture thereof. An increasing amount of CO₂ leads to higher absorption within the body, whereas an increasing amount of O₂ increases the foam stability.

The diameter of the blood vessels to be treated (being preferably veins) is an important parameter for defining the CO₂/O₂ ratio of gas mixture: For large-diameter veins, it is preferred to use foam with a high ratio of CO₂ because it is easier to metabolize and the foam is still stable because the 1% concentration of polidocanol used for big veins provides enough surfactant activity. For small-diameter veins, it is preferred to use a high ratio of oxygen because it is required to use foam with increased O₂-based stability as the concentration of agent can be as small as 0.1%. As the small-diameter veins are located superficially there is enough time for the O₂ to metabolize before entering the general circulation. For a use in sclerotherapy, it is preferred to use a CO₂/O₂ gas mixture with a ratio of between 20/80 to 80/20. For example, the ratio of the CO₂/O₂ gas mixture may be 20/80, 25/75, 30/70, 35/65, 40/60, 45/55, 50/50, 55/45, 60/40, 65/35, 70/30, 75/25 or 80/20.

The preferred position of the first inlet channel at the top part of the foaming chamber, especially in combination with an outlet channel adjacent to or at the bottom part of the foaming chamber allows a rapid and thorough filling of the foaming chamber with the respective gas or gas mixture.

According to the present invention, the supply of the foaming agent and the aqueous diluent to the foaming chambers can be performed by two ways:

In a first embodiment, the second inlet channel allows the supply of a mixture comprising a foaming agent and an aqueous diluent to the foaming chamber representing the liquid components of the foam to be generated. According to this embodiment the third inlet channel can be dispensed with.

In an alternative embodiment, the second inlet channel is used for supplying the aqueous diluent to the foaming chamber provided together with a third inlet for supplying the foaming agent to the foaming chamber. Hereby, the second inlet channel is preferably provided at the top part of the foaming chamber. This also allows a rinsing of the foaming chamber with the aqueous diluent as initial preparation of the foaming chamber or between two different foaming processes. In this configuration it is preferred that the third inlet channel is preferably provided adjacent to or at the bottom part of the foaming chamber. This has the advantage that the complete volume of the foaming agent is provided in the area of the foaming element, where it can properly be diluted by addition of the diluent and the rotation of the foaming element.

A foaming agent is a material that facilitates the formation of foam. Surfactants are commonly used as foaming agents. When present in small amounts, they reduce surface tension of a liquid or increases its colloidal stability by inhibiting coalescence of bubbles.

In some configurations, the foaming agent can be a mixture of a surfactant with a co-surfactant. Surfactants which are less effective at foam production, may have additional co-surfactants added to increase foaming. In which case, the co-surfactant is referred to as the foaming agent. These are surfactants used in lower concentration in a detergent system than the primary surfactant, often the cocamide family of surfactants.

Examples of foaming agents that can be used for preparation of the microfoam include, without limitation, phospholipids such as phosphatidyl choline; proteins such as albumin; fatty acid alcohols such as polidocanol; sulfate esters of fatty alcohols such as sodium tetradecyl sulfate (Morrhuate sodium); sugar esters, such as sucrose stearate, sucrose distearate or sucrose tristearate; block copolymers such as Pluronic F-68 or Pluronic P-123; zonyl fluorosurfactants; fatty acids, such as stearic acid or oleic acids and their salts such as sodium stearate or potassium oleate.

In some embodiments, polidocanol is used as foaming agent. Polidocanol is a synthetic fatty alcohol (hydroxy-polyethoxy-dodecane) with detergent activity acting by a mechanism known as protein theft denaturation, in which an aggregation of detergent molecules forms a lipid bilayer that disrupts the cell surface membrane causing endothelial cell death. Polidocanol causes fibrosis inside varicose veins, occluding the lumen of the vessel, and reducing the appearance of the varicosity. It has therefore been used to treat varicose veins, hemangiomas, and vascular malformations.

With the other necessary ingredients such as the aqueous diluent and a gas or gas mixture and in the form of a controlled, dispensed microfoam, it can be administered intravenously in the treatment of varicose veins, hemangiomas, and vascular malformations.

The aqueous diluent provides the aqueous solvent required for the micelle formation. Examples of aqueous diluents that can be used for preparation of the microfoam include, without limitation water, isotonic saline, citrate buffer, phosphate buffer, acetate buffer, histidine-lactate buffer, tromethamine-gluconate buffer, aspartate buffer, or sclerodex (hypertonic saline in combination with dextrose). Particularly, the microfoam comprises polidocanol as foaming agent and water or isotonic saline as diluent. Such a microfoam generated with O₂, CO₂, and preferably a mixture thereof, is especially suited for the use in sclerotherapy.

The reaction vessel may be characterized by a reaction unit body defining the foaming chamber that further has a second outlet channel that is in fluid communication with the said foaming chamber for removing waste material from the foaming chamber whereby the second outlet channel is preferably provided adjacent to or at the bottom part of the foaming chamber.

The use of the second outlet channel dedicated to waste removal enables a purification of the foaming chamber. As a result, the reaction vessel can be used for one or more further foaming processes with preferably different foaming parameters.

Since the waste, which is mostly given by residual foam or liquid, is mostly concentrated at the bottom of the foaming chamber, it is preferred to provide said second outlet channel adjacent to or at the bottom of the foaming chamber. Furthermore, rinsing with the diluent from the top of the foaming chamber will accumulate the waste material at the bottom part of the foaming chamber.

In a preferred embodiment, the reaction unit body of the reaction vessel of the invention further has a deflection element for forced deflection generating a downward fluid motion during the foaming process in direction to the rotating foaming element, being preferably situated at the bottom part of the foaming chamber.

The term “deflection element” as used herein, is defined as an element that forces the fluid stream within the foaming chamber downwards in direction to the rotating foaming element. It has been found out that a classical stirring process as generated to the rotating foaming element can induce the formation of several layers, so that a layer of thick foam layer at the bottom is covered by a layer with large-pored foam or even residual liquid. The regeneration of these layers hinders the foaming process and an early layer-generation within the foaming process can even prevent the generation of a microfoam. As the inventors found out, a forced deviation of the material by which “fresh material” from above comes into the reach of the rotating foaming element will breaks up the layers and even prevents any layer generation and will thereby allow the rapid and complete conversion of the starting material into a high quality microfoam.

Expediently, the reaction vessel of the present invention has a reaction unit body which is a sealed reaction unit body and/or a sterile or sterilizable reaction unit body. A “sealed reaction unit body” as understood herein, is a reaction unit body for which the claimed inlet and outlet channels represent the only openings, so that the remaining body surface is water- and gas-tight thereby enabling a controlled and sterile foam preparation.

In an alternative embodiment, the reaction vessel has an unsealed reaction unit body, allowing a gas exchange with the environment. In this embodiment it is preferred to perform the foaming process under a permanent positive pressure in order to create a sterile microfoam.

The sealed reaction vessel can be provided by a vessel which is formed in one piece. Alternatively, the reaction vessel may contain a non-detachable lid. The term “non-detachable lid” as used herein refers to a lid that may not be detached by hand without causing mechanical damage to the reaction vessel. Expediently, such a lid is attached to the vessel by secure bonding or heat sealing.

In an alternative embodiment, the sealed reaction vessel can be provided by a vessel which is formed by two or more pieces. Accordingly, the reaction vessel may consist of an open receptacle closed by a detachable lid.

The reaction vessel can be sterilized to be used for medical purposes and as a result can be also provided as a sterile reaction vessel.

In a preferred embodiment, the rotatable foaming element is configured as a disc with mixing features suitably shaped for inducing a turbulent stream and located around an outer perimeter of said disc, whereas the mixing features comprise one or more of the following: blades, rings, teeth, fins, rips, wings, arms, fingers, perforated plates, grid structures, or a ring-shaped helical spring.

A “disc” as understood herein is a flat circular structure which can possess a central buckle. The buckle is preferably conical-, frustoconical- or dome-shaped. At its outer perimeter the disc is equipped with mixing features which, when set into ration, can induce a turbulent flow of the components (i.e., the mixture of the foaming agent and the aqueous diluent) within the foaming chamber leading to their thorough mixing and ultimately to the foam generation.

Expediently, the disc is rotationally symmetric to allow a regular low-wear rotation without imbalance.

The mixing features are preferably arranged radially.

In a further embodiment the mixing features define a circumferential gap between the rotatable foaming element and the inner surface of the bottom or the side walls of the foaming chamber.

Hereby, it is preferred that the distance between the lower margin of the rotatable foaming element and the bottom of the foaming chamber is less than 1 mm, preferably less than 0.75 mm and more preferably equal to or less than 0.5 mm. The distance can be thus 0.5, 0.4, 0.3 or 0.1 mm.

Hereby, it is preferred that the distance between the peripheral margin of the rotatable foaming element and the sidewall of the foaming chamber between 0.01 and 5 mm, preferably between 0.5 and 3 mm, more preferably between 0.75 mm and 2.5 mm and even more preferably between 1.0 and 2.0 mm. As an example, the distance can be 0.02, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0 or 4.5 mm.

Preferably, the rotatable foaming element of the reaction vessel is configured to be magnetically driven by a magnetic stirrer located outside the foaming chamber, whereby the magnetic stirrer is located preferably beneath the bottom part of the foaming chamber. Hereby it is preferred that the rotatable foaming element is a disc.

As a consequence, the rotatable foaming element is disposed in the sterile environment of the foaming chamber, so that no further element needs to be introduced from the outside. Since the magnetic stirrer does not enter into the foaming chamber, the risk for contamination of the foam is virtually excluded.

It will be appreciated that according to the present invention, the rotatable foaming element is configured as a magnetic disc or has at least one magnetic member to be magnetically driven by a magnetic stirrer device beneath the foaming chamber.

The foaming element may be driven by at least one rotating magnet or an assembly of electromagnets in the magnetic stirrer device. It is further conceivable that the magnet is rotated by a set of coils that generate a rotating magnetic field. The magnets may be integrated or permanently fixated in the foaming element, or they may be detachable. The magnetic force, induced by the magnets may be strong enough to restrict the foaming element in its lateral movement while rotating by their magnetic interaction with the magnetic stirrer device.

Preferably, it may be provided that at least two or at least three or at least four or exactly four magnets are incorporated into the foaming element, particularly in a circumference of the foaming element for creating a magnetic coupling with at least three or at least four corresponding magnets on a drive shaft of the magnetic stirrer device.

By creating said magnetic coupling the transferring of torque by the magnetic field may advantageously be reinforced or improved. The magnets on the drive shaft may correspondingly be arranged in a circumference around the drive shaft.

In some configurations the rotatable foaming element may contain one or more elements made from soft iron, nickel, or copper, and preferably a core made from soft iron. Soft iron is a material which can be magnetized in the magnetic field build up by the external actuator.

The rotatable foaming element may be configured to have a centrally located bore, which is preferably open at one end for receiving the positioning member.

Expediently, the positioning member is a centrally or eccentrically located shaft on the bottom of the foaming chamber and preferably has a cylindrical side wall with a rounded or angular upper corner.

The centrally located shaft, which thereby represents a central axle for the rotatable foaming element is preferably centrally located in the foaming chamber, allowing constant gaps between the outer perimeter of the disc and the adjacent walls of the foaming chamber. These constant gaps when exhibiting proper dimensions also allow an optimal foaming process.

Based on the second alternative of an eccentrically located shaft, the rotatable foaming element is also eccentrically located in the foaming chamber, which was found to further enhance the turbulent flow. Being eccentrically located in this context may describe that the predefined axis does not equal an imaginary axis which extends from the upper area towards the lower area through the geometrical center or centroid of the volume of the foaming chamber in a way that is parallel to the direction of gravity when the apparatus is placed on a plane surface. Said plane surface may be perpendicularly aligned to the direction of gravity.

The rotatable foaming element may be simply mounted onto said positioning member or otherwise may be mounted with a bearing.

It is further conceivable that the positioning member is configured as a stationary axis located and/or mounted at the bottom for the holding and preferably comprises a bearing, particularly axial bearing, of the foaming element. In other words, the stationary axis may hold the foaming element or allow an attachment of the foaming element to the stationary axis and may further comprise a bearing, particularly an axial bearing. The axis may particularly be a vertical axis that is suitable to limit a movement of the foaming element in all horizontal directions. The foaming element may comprise flexible material and may further comprise a hole that is slightly smaller in its diameter than the diameter of the stationary axis to allow for a press fit of the stationary axis in the foaming element. The press fit may be strong enough to restrict or fully prevent a translatory movement of the foaming element during the foaming process. The stationary axis may bring the technical effect of a smooth and consistent rotation of the foaming element.

In the context of the movement of the foaming element, the term translatory movement may define a movement of the foaming element along the predefined axis, i.e. perpendicular to the rotary motion of the foaming element.

In another embodiment, the stationary axis is provided as a through axis, extending from the bottom to the top of the reaction chamber.

According to one embodiment, the reaction vessel may further comprise a restriction member for restricting an upward movement of the rotatable foaming element. This restriction member prevents the foaming element to be fall off the positioning member during transport or handling.

This restriction member is preferably a rod-shaped element extending preferably from the top part of the foaming chamber.

To be able to restrict the movement, the restriction member may comprise at least one structural component like a wall or protrusion that blocks the translatory movement of the foaming element. However, a certain amount of translatory movement may be allowed as long as the foaming element does not leave the positioning member. Therefore, the translatory movement, for example in direction of the predefined axis, may be allowed but restricted: preferably the movement is allowed only for a distance less than the length of the position member.

The restriction member may be part of the upper area or a separate component of the foaming chamber. It is also conceivable that the restriction member is the top part itself. Furthermore, the restriction arrangement may be attached to the upper part or other parts of the foaming chamber.

According to an advantageous further development of the invention, it can be provided that the restriction member has a protrusion, particularly a spike, protruding from the upper part of the foaming chamber for restricting the movement of the foaming element towards the upper area and/or off the predefined axis. The protrusion may be made of plastic and/or may have a convex shape in direction to the foaming element. In other words, the protrusion may be described as an extension of material extending from the upper area. It may extend fully towards the foaming element to touch the foaming element directly or it may leave a space of at most 20, preferably at most 15, more preferably at most 10 and even more preferably at most 5 mm. As an example, the space can be 1, 2, 3, 4, 5, 6, 7, 8, 9, 10, 11, 12, 13, 14, 15, 16, 17, 18 or 19 mm.

It is further conceivable within the scope of the invention that the restriction member has a stop plate for restricting the movement of the foaming element towards the upper area and/or off the predefined axis.

The stop plate may be arranged at the end of the aforementioned protrusion. The stop plate may touch the foaming element directly in which case the stop plate may be rotatable along the predefined axis of the foaming element, or it may be arranged at a distance of at most 5 mm to the foaming element.

It is conceivable within the scope of the invention that the deflection element is a deflector plate or deflector body located in the foaming chamber and/or a propellor provided on the rotating foaming element.

The deflector plate may be formed as an even or bent plate, which is oriented in an angle between 10 and 80 degrees to the horizontal, i.e. the bottom plate of the foaming chamber. Given a uniform direction of rotation it builds an acute angle with the entry side of the foaming element, so that the rotation of the foaming element forces the fluids into a downward movement towards the foaming element to be directly transported to the mixing features.

For example, the angle of the deflector plate can be 10, 20, 35, 40, 45, 50, 55, 60, 65, 70, 75 or 80 degree. The angle of the deflector plate is preferably between 40 and 50 degree.

The deflector body is a body with a beveled side, which is oriented in an angle between 10 and 80 degrees to the horizontal, i.e. the bottom plate. Given a uniform direction of rotation it builds an acute angle with the entry side of the foaming element, so that the rotation of the foaming element forces the fluids into a downward movement towards the foaming element to be directly transported to the mixing features.

For example, the angle of the beveled side of the deflector body can be 10, 20, 35, 40, 45, 50, 55, 60, 65, 70, 75 or 80 degree. The angle of the beveled side of the deflector body is preferably between 40 and 50 degree.

In a preferred embodiment of the invention, the deflector body is a circular ring segment of trapezoidal basic structure with a beveled side towards the bottom of the foaming chamber and a bottom horizontal side situated above the rotatable foaming element to provide a gap between the deflector body and the upper margin of the rotatable foaming element, whereby circular ring segment is preferably attached to the cylindrical wall of the foaming chamber, having more preferably a radial ring thickness that substantially covers the mixing features of the rotatable disc.

Based on a foaming chamber with circular cross-section this trapezoidal form turned out to be of advantage. It is to note that the beforementioned circular cross-section denotes to a horizontal cross-section of the foaming chamber.

Hereby, it is preferred that the gap between the deflector body and the upper margin of the rotatable foaming element is less than 2.0 mm, preferably less than 1.5 mm more preferably less than 1.0 mm. As an example, the distance can be 0.1, 0.1, 0.2, 0.3, 0.4, 0.5, 0.6, 0.7, 0.8, 0.9 or 1.0 mm. A certain gap is required to suck sufficient amount of material into the rotating foaming element. However, by increasing the distance, the turbulence of the turbulent flow is reduced and thereby also the efficacy of the foaming process.

According to an advantageous further development of the invention, it can be provided that the first outlet channel is situated under the bottom horizontal side of the deflector body.

This arrangement allows the extraction of high-quality microfoam which is found to be concentrated in this specific area of the foaming chamber. Furthermore, a retrieval of microfoam from this area directly forces the entry of above lying less dense foam material in the zone of highest flow turbulence, thereby maintaining the generation of microfoam.

It shall be underlined that the, possibly magnet-driven, rotation element is not a necessary embodiment of the inventive concept. A rotation element might be avoided, or the rotation element might be an additional foaming means. E.g., there might be a foaming mechanism that creates form by pressing a foaming sclerosant liquid through a filter, so that foam is created. However, even in this case, a rotating element in the foam(ing) chamber might be used to maintain the foam in a certain window of properties.

The reaction vessel may further comprise a gas distributor system, which helps to evenly distribute the gas or gas mixture entering from the first inlet channel into the foaming chamber. This has the advantage that the foaming chamber is filled more homogenously with the gas/gas mixture required for the foaming process and furthermore enables a complete filling without “dead zones” filled with the prior gas.

The gas distributor system is preferably provided as a deviation plate or body located in the foaming chamber beneath the first inlet channel to induce a lateral deflection of the gas stream entering the foaming chamber. In this arrangement the deviation plate or body represents a barrier for the inflowing gas or gas mixture and deflects it lateral to the walls and/or the lower parts of the foaming chamber.

In a further preferred embodiment said gas distributor system is provided by a semispherical-, conical-, frustoconical- or dome-shaped top part of the foaming chamber with the first inlet channel at the top enabling a gas flow which is uniformly distributed over the horizontal cross-section of the foaming chamber. As the inventors found out, the above-described form of the upper part enables a uniform gas distribution.

The gas distributer system can also be realized by a mesh or net through which the gas is guided before or after entering the foaming chamber.

According to an advantageous further development of the invention, it can be provided that the surface of an upper section of the foaming chamber is coated with a hydrophobic or superhydrophobic agent or is a morphologically structured (super) hydrophobic surface such as a superamphiphobic, a slippery liquid-infused porous surface (SLIPS), or a liquid-impregnated surface (LIS).

Due to this (super) hydrophobic surface, the adhesion of liquid components will be diminished or even abolished in the upper, “non-functional part” of the foaming chamber so that virtually the complete material is supplied to the rotating foaming element at the bottom, resulting in a highly efficient foaming process. Furthermore, the foaming chamber can be cleaned more easily after a foaming generation.

In some embodiments, the reaction vessel comprises a pressure compensation device for compensating for pressure fluctuations in the pressure within the foaming chamber.

As a result, the pressure compensation device reduces an overpressure and also increases a negative pressure to maintain the prior reaction pressure within the foaming chamber, thereby the pressure compensation device stabilizes the pressure within the foaming chamber.

In one configuration, the pressure compensation device could be provided by a pressure compensation vessel which is fluidly connected to the foaming chamber. In case of an overpressure within the foaming chamber the gas will flow freely from the foaming chamber to the connected pressure compensation vessel and in case of negative pressure, the gas will flow freely in the opposite direction, namely from the connected pressure compensation vessel to the foaming chamber.

This technical solution has the advantage that it works within a closed system and thereby retains the sterility of the foaming chamber.

In a second aspect, the inventive concept relates to a cartridge containing:
a. a reaction vessel according to the present invention,
b. a foam extraction port containing a valve and arranged accessible from an outside of the cartridge for extraction of the produced microfoam, and
c. a fluid conduit coupling the first outlet channel of the foaming chamber with the said foam extraction port.

The reaction vessel represents the core element of the cartridge as responsible element for the foam generation. This reaction vessel being provided within the cartridge has a fluid conduit for fluidly coupling the first outlet channel of the foaming chamber with the said extraction port. This is the simplest cartridge design, preferably to be used with a reaction vessel that is prefilled with the foaming agent, the aqueous diluent and the gas or gas mixture.

In a further possibility it may be provided that the foaming chamber is at least partly or fully arranged inside a cartridge body of the apparatus. The cartridge body may be configured as a housing. It is possible that the cartridge body is configured to be watertight. It is further possible that the cartridge body comprises at least one or exactly one of the following materials: plastic, metal, rubber, wood, cardboard, paper and ceramics. The cartridge body may also comprise a sterilizable surface.

The incorporation of the reaction vessel into a cartridge together with a network of fluid conduits allow the automatization of the foam generation process by installing said cartridge in an automated device dedicated hereto.

The use of a cartridge has the further advantage that a sterile environment can be provided for the foam production, i.e., the foaming process.

Due to the location of the foam extraction port being accessible from an outside of the cartridge, it allows the extraction of the microfoam. The foam extraction port constitutes an interface between the cartridge an its environment.

The foam extraction port contains a valve for secure extraction of the microfoam from the cartridge. This valve may be a one-way valve ensuring the extraction of the microfoam without compromising the sterile environment in the foaming chamber. It may alternatively be a solenoid valve that is driven and controlled by an electric circuit that may be connected and/or controlled via a user interface. Preferably, said valve is a Luer taper or a Luer lock for mating with the tip of a syringe. In one configuration, the foam extraction port may comprise a self-sealing valve that opens by connecting an extraction device, preferably a syringe.

The fluid conduit enables the transport of the microfoam from the first outlet of the foaming chamber to the foam extraction port. Expediently the fluid conduit is as water and gas-tight conduit. The fluid conduit can be a tube, a channel or a pipe. It may comprise or be made from plastic, rubber or metal. Preferably, the fluid conduit is a flexible tube.

In a further embodiment, the fluid conduit comprises a particle filter, being preferably a sterile filter, in order to filter the microfoam transported to the foam extraction port.

For the case that the cartridge contains a further inlet channel(s) for the supply of the aqueous diluent and foaming agent or a mixture thereof, the cartridge may further contain:
a. a waste container defining a waste chamber containing an inlet for receiving the waste material from the foaming chamber,
b. a fluid conduit coupling the second outlet channel of the foaming chamber through a valve with the said inlet of the waste container, and
c. the waste container optionally contains an outlet for pressure release.

In a preferred embodiment, the cartridge also contains a waste chamber for receiving the waste material from the foaming chamber. The term “waste material” as used herein refers to a residue of the used liquid composition or to a residue of the microfoam generated in the foaming chamber that is not extracted. It also includes the washing liquid generated by rinsing the foaming chamber with a washing solution being preferably the aqueous diluent used for generating the microfoam. By removing the waste material and, if applicable, also the washing liquid, the foaming chamber is made available for a new foaming reaction. This may be useful in a multiphasic foaming process wherein at least two microfoam preparations of different concentrations and/or different gas ratios are produced. With regard to the treatment of varicose veins, the generation of different foams may be necessary for a treatment of differently sized veins. It is furthermore also useful in a multiphasic foaming process wherein a discharge of at least two microfoam preparations of the same concentrations and gas ratios are produced, insofar a residual foam might impede the production of the new foam.

Furthermore, it is advantageous to provide the fluid conduit with a valve to control the flow of the waste material from the foaming chamber to the waste chamber. This valve should be closed during the foaming process and the foam extraction phase and should be specifically opened for removal of the waste.

In an optional embodiment, the waste chamber contains an outlet for pressure release. The pressure release allows the reduction of an over pressure generated by the waste material entering the waste chamber. In a preferred embodiment the outlet comprises a particle filter, being preferably a sterile filter, in order to filter the gas which is emitted from the waste chamber and/or prevents the discharge of foam.

In some embodiments the inlet of the waste container is positioned in the top part of the waste container, preferably as the upper aperture of a hollow shaft positioned preferably in the center of the waste chamber. This elevated position in relation to the bottom of the waste chamber is a safety measure since the waste material will flow downwards to the bottom of the waste chamber so that in case of an erroneous back flow, only gas will be transported back to the foaming chamber.

Furthermore, it is advantageous for the cartridge that the valve for controlling the flow of the waste material into the waste chamber is located on the outer surface or accessible from an outer surface of the cartridge and preferably can be controlled by exerting an external pressure on said valve. The aforementioned advantages and effects become particularly effective if the cartridge is inserted in an automated device allowing an automatic control of said valve.

The provision of a valve located on the outer surface or accessible from an outer surface of the cartridge conduits allow the automatization of the foam generation process by installing said cartridge in an automated device dedicated hereto.

In some configurations, the inner surface of the waste chamber of the cartridge is coated with a defoaming agent or has a morphologically structured defoaming inner surface such as an anti-foaming surface such as a superamphiphobic, a slippery liquid-infused porous surface (SLIPS), or a liquid-impregnated surface (LIS). This has the advantage that the foam as component of the waste material will be eliminated and converted into the liquid portion which is collected at the bottom of the waste chamber and the gas portion which can be emitted by the pressure release. As a result, the waste chamber can collect much more waste material.

In some embodiments, the cartridge further contains:
a. a gas supply port containing a sealing member and arranged accessible from an outside or located on the outer surface of the cartridge for supply of a gas or gas mixture.
b. a fluid conduit coupling the said gas supply port and the first inlet channel of the foaming chamber.

The gas supply port allows the supply of the gas or gas mixture required for the foaming process from a gas (mixture) source located outside of said cartridge.

The fluid conduit enables the transport of the gas or gas mixture from the gas supply port to the first inlet of the foaming chamber. Expediently the fluid conduit is as gas-tight conduit. The fluid conduit can be a tube, a channel or a pipe. It may comprise or be made from plastic, rubber or metal. Preferably, the fluid conduit is a flexible tube.

In a preferred embodiment, the fluid conduit comprises a particle filter, being preferably a sterile filter, in order to filter the gas or gas mixture transported to the foaming chamber.

The provision of a gas supply port located on the outer surface or accessible from an outer surface of the cartridge conduits allow the automatization of the foam generation process by installing said cartridge in an automated device dedicated hereto.

It will be appreciated that according to the present invention, the cartridge further contains:
a. a diluent supply port containing a sealing member and arranged accessible from an outside of the cartridge for supply of an aqueous diluent,
b. a fluid conduit coupling the said diluent port and the second inlet channel of the foaming chamber,
c. a foaming agent reservoir, being preferably a syringe, enabling a fluid tight seal with the third inlet channel and preferably also with a foaming agent reservoir port.

The diluent supply port allows the supply of the aqueous diluent required for the foaming process from an aqueous diluent source located outside of said cartridge. Preferably the aqueous diluent is water or isotonic saline solution. Expediently, these solutions are provided in a externally located infusion bag and/or bottle.

The fluid conduit enables the transport of the diluent from the diluent supply port to the second inlet of the foaming chamber. Expediently the fluid conduit is as water-tight conduit. The fluid conduit can be a tube, a channel or a pipe. It may comprise or be made from plastic, rubber or metal. Preferably, the fluid conduit is a flexible tube.

In a preferred embodiment, the fluid conduit comprises a particle filter, being preferably a sterile filter, in order to filter the aqueous diluent transported to the foaming chamber.

The foaming agent reservoir can hold the foaming agent required for the foaming process which has to be diluted with the aqueous diluent in order to generate the final foaming solution.

In one configuration, the foaming agent reservoir is prefilled with the selected foaming agent to be transported through the fluid conduit to the third inlet channel of the foaming chamber in order to be diluted with the aqueous diluent within the foaming chamber.

In an alternative configuration, the foaming agent reservoir can be filled via the foaming agent reservoir port.

Preferably, the foaming agent reservoir is a syringe installed in the cartridge which can be easily filled by external filling the syringe and thereby pulling the plunger and emptied in a controlled manner by pushing the plunger over a defined distance.

The provision of a diluent supply port located on the outer surface or accessible from an outer surface of the cartridge conduits allow the automatization of the foam generation process by installing said cartridge in an automated device dedicated hereto.

In some embodiments, the cartridge further contains:
a. a foaming agent supply port containing a valve and arranged accessible from an outside of the cartridge for supply of a foaming agent,
b. a fluid conduit coupling the said foaming agent supply port with the foaming agent reservoir port and with the third inlet channel of the foaming chamber, wherein said fluid conduit contains a valve which is positionable to allow a fluid communication of the foaming agent reservoir port with the foaming agent supply port or alternatively of the foaming agent reservoir port with the third inlet channel, and wherein said valve is preferably switchable by exerting an external pressure on the cartridge.

The foaming agent supply port allows the supply of the foaming agent required for the foaming process from a foaming agent source located outside of said cartridge. Preferably the foaming agent is a sclerosing agent such as polidocanol. Expediently, the foaming agent is provided by a syringe filled with the foaming agent and injected through the valve of the foaming agent supply port.

The fluid conduit enables in the transport of the foaming agent from the foaming agent supply port to the foaming agent reservoir port and also enables the transport from the foaming agent reservoir to the third inlet channel of the foaming chamber. Expediently the fluid conduit is as water-tight conduit. The fluid conduit can be a tube, a channel or a pipe. It may comprise or be made from plastic, rubber or metal. Preferably, the fluid conduit is a flexible tube.

It will be appreciated that the two aforementioned liquid flows work in a complementary manner, insofar the flow from the foaming agent from the foaming agent supply port to the foaming agent reservoir port excludes the transport from the foaming agent reservoir to the third inlet channel of the foaming chamber and vice versa.

This can be accomplished by the valve contained within the fluid conduit and which is positionable to allow a fluid communication of the foaming agent reservoir port with the foaming agent supply port or alternatively of the foaming agent reservoir port with the third inlet channel, and wherein said valve is preferably switchable by exerting an external pressure on the cartridge.

The provision of a foaming agent supply port located on the outer surface or accessible from an outer surface of the cartridge conduits allow the automatization of the foam generation process by installing said cartridge in an automated device dedicated hereto.

The foaming agent reservoir, being preferably a syringe, may be capable of storing at least 4 mL of foaming agent solution.

Generally, it is preferred that the cartridge further contains means for uniquely identifying the cartridge and/or its properties, wherein said identification means is preferably selected from an RFID tag, a barcode, a data matrix code, a QR code or a similar code.

The unique identifier may contain at least one of the following information: a compatibility information, a type information, an authenticity information, an expiry data of the cartridge, production date, status of use, a lot and/or serial number. The identification means may be provided via a RFID chip in which case the automated device may further comprise a RFID reader. RFID may in the context of the invention stand for radio-frequency identification. In case of a bar code, a data matrix code or a QR code, the automated device may comprise a respective code reader.

It is also advantageous if the cartridge of the present invention is configured as a disposable cartridge for the use in a medical device and/or a laboratory device, and particularly in the automated device according to the present invention.

The cartridge being configured as a disposable cartridge may bring the technical effect that it prevents the spill over of any contamination from one patient to another.

In some embodiments, the cartridge comprises a pressure compensation device for compensating for pressure transients in the pressure within the foaming chamber.

As a result, the pressure compensation device reduces an overpressure and also increases a negative pressure to maintain the reaction pressure within the foaming chamber.

The pressure compensation device can be realized by a pressure sensor in the gas supplying conduit system arranged to open a pressure release valve in case of overpressure or opening the valve for increasing the gas flow in case of negative pressure.

In an alternative embodiment, the pressure compensation device could be provided by the provision of a pressure compensation vessel given within the cartridge which is fluidly connected to the foaming chamber. In case of an overpressure within the foaming chamber the gas will flow freely from the foaming chamber to the connected pressure compensation vessel and in case of negative pressure, the gas will flow freely from the connected pressure compensation vessel to the foaming chamber.

Both technical solutions have the advantage that they work within the closed system and thereby retain the sterility of the foaming chamber.

In a third aspect, the invention provides an automated device for generating microfoam containing:
a. a cartridge holder adapted to hold a replaceable cartridge according to the invention,
b. a magnetic stirrer situated beneath the cartridge for generating a rotating magnetic field for controlling the rotation of the foaming element around the positioning member.

The aforementioned advantages and effects of the cartridge become particularly effective if the cartridge is used together with an automated device.

In this configuration, the cartridge represents the core element of the automated device as responsible element for the foam generation. The automated device comprises the chemicals required for the foaming process such as gas or gases and the aqueous diluent, and is equipped to deliver the necessary amounts to the cartridge based on the specific parameters selected by the user.

The automated device uses a cartridge holder in order to hold and secure the cartridge in order to deliver the chemicals in a reliable and safe manner.

The automated device can further comprise a magnetic stirrer which is situated beneath the cartridge in a way for controlling the rotation of the foaming element around the positioning member.

The automated device may be used for preparation of medical microfoam of different characteristics, such as defined by different concentrations of the foaming and/or a different gas mixture ratio. The results of such medical foam production processes can be regarded as a pharmaceutical preparation which normally require the preparation by a certified pharmacist in order to be used in medical applications. However, the use of a validated automated device eliminates the need for a pharmacist so that the clinician itself can prepare the medical microfoam according to his requirements.

The magnetic stirrer may comprise at least one rotating magnet or an assembly of electromagnets or a set of coils for generating the rotating magnetic field. The system according to the invention may comprise a power supply apparatus that provides an electric supply, particularly a supply voltage, for the system. The supply voltage may be in the range from 90 V to 400 V and preferably in the range from 90 V to 260 V.

The automated device may be configured as a stand-alone system so that it can perform its function without any additional devices. It also may comprise a modular design. Furthermore, it may also comprise interfaces for communication and/or configuration of the foaming parameters such as a WLAN interface, wherein WLAN stands for Wireless Local Area Network, or a Bluetooth interface. The system may further comprise a serial, particularly USB interface, preferably in a location that is easily accessible for a user when operating the system. It is also conceivable that the system comprises an NFC (Near Field Communication) or a RFID (Radio Frequency Identification) interface, for example to allow an identification of the user or the configuration of the foaming parameters.

It is possible that the system according to the invention comprises at least one storage compartment for at least one or at least two or at least three containers, wherein particularly each of the containers store an amount of a consumable. The consumables (for instance a liquid composition, a diluent, an agent solution, a gas or a mixture of gases) stored by the storage compartment may be sufficient for at least 20, preferably at least 40, particularly at least 50 foaming processes in a row.

It is further conceivable that the automated device comprises at least one sensor for detecting a presence of at least one component of the liquid composition or the liquid composition as a whole or the consumables as described above. The at least one sensor may be an optical, an ultrasonic or a weight sensor and may be arranged so that a detection of the presence of the at least one component of the liquid composition or the liquid composition as a whole may be performed by monitoring at least one pipe of the system in which at least one of said fluid or fluids may be transported.

The components of the liquid composition may be selected as at least one from the following: a foaming agent and a suitable solvent, i.e. an aqueous diluent. The consumable may be selected as at least one from the following: a liquid composition, a diluent, a foaming agent, a gas or a mixture of gases, wherein the diluent may be sterile water (particularly distilled water) or physiological saline, the foaming agent may be polidocanol, sodium tetradecyl sulfate, albumin or phospholipids and the at least one gas may be O₂ or CO₂.

It will be appreciated that according to the invention, the cartridge holder of the automated device contains a gas feed port that is configured to fluidly engage with the gas supply port of the cartridge in the holder, thereby providing a gas-tight seal of both ports, wherein the gas feed port of the automated device preferably contains a safety valve allowing a gas supply only for a cartridge being properly locked in the cartridge holder.

Expediently, the cartridge holder of the automated device contains a diluent feed port that is configured to fluidly engage with the diluent supply port of the cartridge placed in the holder, thereby providing a fluid-tight seal of both ports, wherein the diluent feed port or the system preferably contains a safety valve allowing a diluent supply only for a cartridge being properly locked in the cartridge holder.

In some configurations, the automated device contains a sensor that is configured to detect within the cartridge the presence or flow of a liquid in the fluid conduit coupling the diluent port and the second inlet channel of the foaming chamber, whereby said sensor is preferably a capacitive sensor, optical sensor or an ultrasound sensor.

Preferably, the sensor is provided beneath the cartridge in a position underneath or near said fluid conduit in order to be in close proximity to said fluid conduit.

In some embodiments, the cartridge holder of the automated device contains one or more guiding elements for proper insertion of the cartridge within the cartridge holder, wherein the guiding elements preferably contains at least two retractable members arranged at the bottom of the cartridge holder and mating with respective orifices of the cartridge housing.

In some embodiments, the cartridge holder of the automated device contains a fastening mechanism configured to fasten the cartridge to the cartridge holder, whereby the fastening mechanism preferably contains one or more members that are arranged in the lateral wall of the cartridge holder and can be extended to engage with respective features of the cartridge housing.

It is appreciated that according to the invention, that the automated device contains a gas supply system containing:
a. at least one gas port for connection to at least one gas container, being preferably a gas bottle,
b. at least one gas conduit connecting the at least one gas port to the gas supply port via a valve for regulating the gas flow;
c. optionally a pressure compensation device for compensating for pressure transients in the pressure within the foaming chamber.

The gas supply system delivers the gases from the gas container(s) to the cartridge.

The gas conduit enables the transport of the gas from the at least one gas container to the gas supply. Expediently the gas conduit is as gas-tight conduit. The gas conduit can be a tube, a channel or a pipe. It may comprise or be made from plastic, rubber or metal. Preferably, the gas conduit is a flexible tube.

In an optional embodiment, the gas supply system comprises a pressure compensation device (PCD) for compensating for pressure transients in the pressure within the foaming chamber. As such the PCD reduces an overpressure and also increases a negative pressure to maintain the reaction pressure within the foaming chamber.

The pressure compensation device can be provided by a pressure sensor in the gas supply system arranged to open a pressure release valve in case of overpressure or opening the valve for increasing the gas flow in case of negative pressure.

In an alternative embodiment, the pressure compensation device could be provided by the provision of a pressure compensation vessel which is fluidly connected to the foaming chamber. In case of an overpressure within the foaming chamber the gas will flow freely from the foaming chamber to the connected pressure compensation vessel and in case of negative pressure, the gas will flow freely from the connected pressure compensation vessel to the foaming chamber.

Both technical solutions have the advantage that they work within the closed system and thereby retain the sterility of the foaming chamber.

In some embodiments the gas supply system comprises also a safety valve, which automatically interrupts the gas supply in the event of an impermissible drop in pressure.

In some embodiments, the gas supply system of the automated device contains:
a. two gas ports for connection to two gas containers containing different gases, preferably installed in the automated device and being more preferably an O₂ gas container and a CO₂ gas container,
b. a gas channel network between the two gas ports and the gas feed port,
c. a gas mixing device for mixing the two different gases in a selectable ratio.

This configuration of the gas supply system provides a mixture of gases and deliver it via a gas channel network from the gas containers to the cartridge.

In a preferred embodiment, the gas supply system contains a gas container providing oxygen and a gas container providing carbon dioxide.

The gas channel network enables the transport of the gas from the two gas containers installed at the two gas ports to the gas feed. Expediently the gas channel network is gas-tight. The gas channel network can be built from tubes, channels or pipes. It may comprise or be made from plastic, rubber or metal. Preferably, the gas channel network is made from flexible tubes.

In this embodiment of the gas supply system, the gas derived from the two gas containers is mixed in the gas mixing device in a selectable gas ratio and then further transported to the gas feed port of the automated device. Upon proper positioning of a cartridge of the invention, the gas mixture is further transported via the gas supply port of the cartridge and the gas conduit to the foaming chamber for pre-equilibration of the chamber, for the foaming procedure or for post-foaming procedures.

The gases may be provided from independent, preferably consumable, containers and may be mixed according to the configured gas mixture ratio. The configuration of the gas mixture ratio may be provided by a user via a user interface. The gas delivery may comprise at least one sensor for determining an amount of gas to provide the mixture of gases. In case the gas container comprises means for storage such as a canister or bottle the amount of gas may be determined by using a weight sensor such as a load cell for measuring the weight of the canister or bottle. It is further conceivable that an ultrasound sensor is used for determining the amount of gas in the means for storage. Another possible way of enabling the mixing of the gasses according to a desired ratio would be to use a gas flow meter for determining an amount of gas volume per time unit. Another alternative would be to use a pressure sensor to calculate the remaining amount of gas. The sensors described above may be part of the automated device and specifically the gas supply system according to the invention.

In some embodiments, the automated device contains a diluent supply system contains:
a. a diluent port for connection to a diluent container, preferably installed in the automated device, and
b. a liquid conduit connecting the diluent port to the diluent feed port via gravitation or a pump capable of transporting the diluent from the diluent container to the diluent feed port.

The diluent supply system allows the supply of the aqueous diluent from the diluent container, preferably installed in the automated device, to the diluent feed port. Upon proper installation of a cartridge of the invention, the diluent is further transported via the diluent supply port of the cartridge and the liquid conduit to the foaming chamber for pre-equilibration of the chamber, for the foaming procedure or for post-foaming procedures (such as cleaning).

The transportation of the diluent can be performed by gravitation. In case of an infusion bag, this can be accomplished by installing the bag and/or bottle at an enhanced position.

In an alternative configuration, the diluent transport is accomplished by a pump installed in the diluent supply system. Preferably, a peristaltic pump is used for this purpose.

In a further alternative, the diluent transport is accomplished by a pressure means installed in the diluent supply system and exerting a controlled pressure on a flexible diluent container.

Preferably, the aqueous diluent is water or isotonic saline solution. Expediently, these solutions are provided by an infusion bag filled with water or isotonic saline solution, whereby said bag is preferably installed in the automated device.

The liquid conduit enables the transport of the diluent from the diluent port to the diluent feed port. Expediently the fluid conduit is as water-tight conduit. The fluid conduit can be a tube, a channel or a pipe. It may comprise or be made from plastic, rubber or metal. Preferably, the fluid conduit is a flexible tube.

In a preferred embodiment, the fluid conduit comprises a particle filter, being preferably a sterile filter, in order to filter the aqueous diluent transported to the diluent feed port.

The automated device may use the diluent from a replaceable container to generate the foaming dilution at different concentrations. The automated device according to the invention may therefore further comprise at least one sensor for determining an amount of diluent that is present in said replaceable container such as a weight or ultrasound sensor. It is also conceivable that the amount of diluent that is transported through the diluent delivery is determined by a flow sensor.

In one embodiment said ports may be provide as a self-sealing transfer ports, particularly in the form of a normally-closed valve. The term self-sealing particularly describes that the transfer port, particularly the normally-closed valve, seals itself to be watertight when no extraction or supply is performed. The term normally-closed may describe that the valve is closed as long as no transferring of the at least one component is performed.

In some embodiments, the automated device contains a syringe-associated delivery system containing a drive unit moving an agitator which can perform at least one of the following actions:
a. detecting the correct connection of the inserted syringe to the foaming agent reservoir port of the said holding member,
b. detecting the filling of the syringe via the foaming agent reservoir port by detecting the position of the plunger,
c. delivering a determined amount of foaming agent to the foaming chamber by pushing the plunger of the foaming agent-containing syringe to a controlled extent,
d. optionally detecting malfunctions of the syringe by determining the amount of pressure to the executed on the plunger of the syringe.

The aforementioned advantages and effects of a syringe being installed in the cartridge as foaming agent reservoir become particularly effective if combined with the above-described configuration of the automated device.

By detecting the correct connection of the inserted syringe to the foaming agent reservoir port of the said holding member, the automated device ensures the proper installation of the foaming agent reservoir which represents an important test after installation of the cartridge within the automated device.

By detecting the position of the plunger, the automated device can detect the filling status of the syringe as filled via the foaming agent reservoir port.

In a subsequent process step, the automated device can push the plunger of the foaming agent-containing syringe to a controlled extent in order to deliver a determined amount of foaming agent to the foaming chamber.

In an optional embodiment, the automated device can detect malfunctions of the syringe by determining the amount of pressure to be executed on the plunger of the syringe.

In some embodiments, the automated device further comprises:
a. a control computing unit, in which the control program is located connected with a user interface for controlling the foaming process, particularly by configuring at least one parameter of the foaming process and/or by initiating the foaming process, the at least one parameter being directly or indirectly selected from the following:
   i. a gas concentration,
   ii. a mixing ratio of the gas mixture,
   iii. a concentration of the foaming agent in the mixture of foaming agent and diluent,
   iv. the total amount of the mixture of foaming agent and diluent, selection of the liquid composition,
   v. the rotation speed and time of the rotatable foaming element,
   vi. the removal of waste material from the foaming chamber,
   vii. the cleansing of the foaming chamber as preparation for a further foaming process,
   viii, the removal of the cartridge from the cartridge holder,
   ix. the time period of gas flow;
   x. definition of ramps for increase or decrease of the rotation speed;
      wherein the automated device is configured to perform the foaming process based on the configured parameter automatically, and particularly the mixing of the gases according to the configured mixing ratio of the gas mixture automatically.

It will be appreciated that the microfoam can be produced automatically by the automated device according to the invention. It can for example be provided that only at least one parameter of the foaming process is manually configured and/or the foaming process is manually started and from then on, the foaming process is performed automatically.

The production may therefore be performed fully or at least partially automatically.

The term “automatic” or “automatically” may refer to the process of producing the microfoam, i.e. the foaming process so that it may describe the fact that no intervention or action by a user is necessary while the microfoam is produced and/or the foaming process is running.

The composition and/or concentration of the gases and the mixing of the gases and the agent solution may, but not necessarily, be done automatically.

It is also possible that the concentration of the gases and the agent solution may automatically be set depending on a selection of predefined presets or scenarios by a user.

The automated device may be configured to perform the foaming process based on the configured parameter automatically, and particularly the mixing of the gases according to the configured mixing ratio of the gas mixture automatically.

For the automatic performance, at least one or multiple predefined process flows may be saved in a non-volatile memory of a control arrangement.

The control arrangement may further comprise a control unit that particularly has a processor or the like and that is configured to perform the following steps:
reading out the at least one predefined process flow, and/or
parametrizing the process flow using the configured parameter, and/or
controlling the rotation of the foaming element and/or controlling the mixing of the gases according to the configured process flow.

The user interface may comprise several components to allow an interaction with the user, such as a screen as well as a graphical user interface on said screen. The user interface may comprise a graphical screen, particularly a touchscreen, preferably with capacitive touch input as the main means of interaction with the user. It may alternatively or additionally comprise mechanical buttons and/or a keyboard and/or a mouse. The graphical screen may have a size of at least 8 inches, particularly at least 10 inches, diagonally. The user interface may also comprise at least one loudspeaker for providing acoustic information or guidance concerning the foaming process. It may also comprise at least one microphone for allowing a voice control of the user interface and/or an optical signaling device, such as LEDs.

Preferably, it may be provided in the context of the invention that the system further comprises:
A control arrangement for initiating a transport of at least one component for the liquid composition to the foaming chamber depending on at least one parameter of the foaming process, wherein at least two or at least three different components are provided, wherein a mixing ratio of the components is set by the at least one parameter and is controlled by the control arrangement,
a user interface to configure the at least one parameter and/or to activate the control arrangement.

The control arrangement may comprise an electric circuit which controls at least one transport element, f.e. a valve, for initiating the transport wherein said valve may be a solenoid valve. The control arrangement may further or alternatively comprise mechanical components such as a pump or a flap for initiating the transport. To control the mixing ratios of the components of the foaming process and to initiate the transport depending on the at least one parameter of the foaming process, the control arrangement may further comprise or be connected to at least one sensor, for instance a weight sensor or flow sensor, that measures the weight of a gas or a pressure sensor to calculate the flow of a gas or a liquid. The control arrangement may further comprise a computer, particularly a microcomputer, to interpret data of the at least one sensor and/or to initiate the transport of the at least one component for the liquid composition. It is also possible that the control arrangement is configured to control the foaming process, particularly a rotation of the foaming element, preferably a rotational speed and/or a starting of the rotation and/or a stopping of the rotation and/or a duration of the rotation. Therefore, the rotational speed and/or the duration of the rotation may be parameters of the foaming process that can be set and/or modified by the control arrangement automatically and/or using a manual configuration by the user interface. It is also possible that the foaming process is performed using different rotational speeds within one single foaming process, so that a change of the rotational speed is controlled by the control arrangement without stopping the rotation. It is also possible that different foaming processes are performed with different rotational speeds.

Furthermore, it may be advantageous within the scope of the invention that the apparatus is configured as a replaceable cartridge for being placed in a cartridge inlet above the stirrer. The system may therefore comprise means to allow for a user initiated de- and attachment of the apparatus, preferably at least partly arranged in said cartridge inlet. It is conceivable that said means are implemented by a release button or lever that may release the apparatus for example by opening hooks that are designed to hold the apparatus.

It is possible that the system further comprises:
A replaceable container for providing a diluent for the liquid composition,
At least one replaceable container for providing a foaming gas or at least two replaceable containers for providing different mixtures of the gases.

If a supply of different mixtures of gases is intended, the gases may be stored in separate containers to then be mixed in a desired ratio. The at least one replaceable container may comprise or be completely made of recyclable material.

Furthermore, it is conceivable that the automated device according to the invention is configured to perform the foaming process automatically and repeatedly as long as the foaming agent solution is available in the foaming agent reservoir. This may be achieved by monitoring the quantity of the foaming agent in the agent reservoir, for example using a sensor. Alternatively, this may be achieved by predefined parameters such as certain time periods. In general, the automated device according to the invention may provide a monitoring system for monitoring the amount of at least one consumable (e.g., gas or gases, foaming agent and/or aqueous diluent). The monitoring system may provide at least one sensor. This may entail the technical effect that the automated device according to the invention can at least partly operate without the need of supervision by qualified personnel. It is possible that the monitoring system outputs a warning to the user via a user interface if one of the consumables falls below a defined threshold. Alternatively, or additionally, the warning may be provided as a sound or a noise. It is further possible that the monitoring system may limit the usage of the apparatus over a period of time or over a number of foaming cycles to ensure sterile conditions in which case an according notification may be provided via the user interface or as a sound or a noise.

Furthermore, it may be provided that the automated device is configured to be used in sclerotherapy or for the therapy of other vascular disorders, wherein the apparatus is particularly configured as a laboratory apparatus.

In a preferred embodiment, the automated device is configured as a mobile system, e.g. equipped with wheels, so that the automated device can be moved to the subject in need thereof. As a result, it could be used locally (e.g. at the bedside) allowing a convenient medical preparation of foam for therapeutic or diagnostic purposes.

It is also optionally conceivable that the automated device according to the invention is configured as a laboratory device for automatically producing the microfoam, preferably a medical microfoam for use in an invasive or minimally invasive medical procedure.

The automated device is suitable for a clinical use and/or the use in a laboratory environment and therefore may comprise corresponding adaptions like a disinfectable housing or the like. The housing may be suitable to withstand cleaning of its outer surface with respective cleaning agents and/or disinfectants such as 4% sodium hypochlorite solution and/or with 70% ethanol.

In a fourth aspect, the invention relates to a method for automatically producing a microfoam comprising the following steps:
a. providing the reaction vessel according to the present invention,
b. optionally flushing the foaming chamber with a selected gas or gas mixture or rinsing the foaming chamber with the aqueous diluent, being preferably saline,
c. introducing the selected gas or gas mixture into the foaming chamber via the first inlet channel,
d. introducing a mixture of the foaming agent and the aqueous diluent into the foaming chamber via the second inlet channel, or alternatively, introducing the aqueous diluent into the foaming chamber via the second inlet channel and the foaming agent into the foaming chamber via the third inlet channel,
e. rotating the magnetic stirrer to rotate the foaming element until a suitable microfoam has been obtained,
f. removal of the obtained therapeutical foam from the foaming chamber via the first outlet channel.

Thus, the method according to the invention brings the same advantages as have been described in detail with reference to the reaction vessel, the cartridge and the automated device.

Furthermore, the method can be adapted for being executed using the inventive automated device and/or the inventive computer program to produce the inventive microfoam. The preceding steps of the inventive method can be carried out one after the other or in any order, whereby individual steps can also be carried out repeatedly.

Simply put, according to the inventive method, the microfoam may be produced from the liquid composition comprising the foaming agent and the aqueous diluent and the gas or gas mixture by utilizing the rotation of the foaming element therein, which rotates around the positioning member as a predefined axis. Furthermore, the duration as one parameter of the foaming process, may be in the range from 10 s to 60 s, particularly 30 s to 50 s. The rotational speed of the foaming element during the foaming process may be between 1000 rpm and 5000 rpm and preferably between 400 rpm and 2000 rpm. It is possible that the foaming process produces an amount of at least or exactly 20 ml of microfoam. The method may further comprise a rinsing of the foaming chamber after and/or before the foaming process is performed. In case a multiphase process is performed, the rinsing may be executed before and/or after each foaming cycle. For the rinsing, a diluent, such as saline, may be used.

Based on 5 ml foaming liquid (=volume of the foaming agent diluted with the aqueous diluent) approximately 40 ml to 45 ml of extractable microfoam can be produced.

Regarding step e. it is preferred to increase the rotation speed during the foaming steps, preferably over several stages with ramps, in which the rotation speed is changed, of between 5 sec, and 20 sec.

A preferred protocol includes the following steps:
Ramp from 0 to 2000 rpm in 10 seconds,
Holding at 2000 rpm for 20 seconds (prefoaming),
Ramp from 2000 rpm to 4000 rpm in 10 seconds,
Holding at 4000 rpm for 40 seconds (main foaming phase),
Maintaining the rpm between 2000 rpm and 4000 rpm for foam extraction for up to several minutes.

Preferably, it may be provided in the context of the invention that the method comprises the following steps:
a. inserting a cartridge according to the present invention into the cartridge holder of the automated device according to the present invention,
b. selecting a foaming agent concentration using a user interface,
c. selecting a ratio for the mixture of two gases using a user interface,
d. Filling the foaming agent reservoir by injecting a foaming agent through the sealing member of the foaming agent supply port,
e. Starting the foam generation process using a user interface so that the system automatically rotates the magnetic stirrer to rotate the foaming element in the foaming chamber of the cartridge until a suitable microfoam has been obtained,
f. Once the foam has been generated removal of the microfoam by extraction from the foam supply port of the cartridge,
g. Optionally initiating a cleansing process of the foaming chamber by using a user interface,
h. Optionally repeating the process according steps a. to g.

The method may further comprise the selection of a system-language via the user interface. Furthermore, the method may comprise the selection of at least one foaming profile using the user interface. Each of the provided foaming profiles may comprise a preset configuration for the agent concentration and/or the gas ratio. Additionally, the method may comprise an output of instructions for the foam production depending on the selected foaming profile via the user interface. The output of the instructions may be performed via a graphical screen or a loudspeaker. The output of instructions may be executed in the previously selected language. The cartridge inlet may be configured to hold the cartridge in place while it is attached. It may further be provided that the cartridge may be released through means of user interaction. For said releasing a clamp, plug or snap connection may be provided. Therefore, the method may further comprise steps of attaching and releasing the cartridge into and out of the cartridge inlet. It is also possible that the method further comprises the step of detecting when a cartridge is connected. It may be further provided that a reading of a cartridge information data set is performed which may comprise at least one of the following points of information: a compatibility information, a type information, an authenticity information, an expiry data of the cartridge, a lot and/or serial number. This may provide the technical effect that fake or already used cartridges may be detected. The cartridge information data set may be provided via a RFID chip in which case the inventive automated device may further comprise a RFID reader. The step that the mixture of gasses and the agent solution concentration is automatically transferred to the foaming chamber according to the configured gas ratio and agent concentration may work as follows: The processes of mixing may be controlled by a programmed sequence depending on the configured gas ratio and agent concentration. A software of the inventive system may calculate necessary volumes, which may then be conveyed via volumetric pumps. The volumes may be determined by the number of steps of the pump drive or the pump time, preferably with each step corresponding to a known volume. A linear pump arrangement may be used for providing the agent. For providing a diluent for the liquid composition, a rotating pump, in particular a peristaltic pump, with a pipe may be used. Both pumps may be driven by stepper motors.

It may be conceivable that the method further comprises:
Initiating a conservation process after the foaming process is performed wherein a rotation of the foaming element is provided, particularly a slower rotation than in the foaming process, to keep the microfoam in a state that allows an application of the microfoam to a patient.

For the initiation of the conservation process an electronic or mechanical trigger may be used that is activated when the foaming process is finished. It is also possible that the conservation process may be initiated after a specific time has passed that was calculated or specified for the duration of the foaming process. The conservation process may be used to maintain the foam quality by extending the time window in which the microfoam is in a state that allows an application of the microfoam to a patient, in other words in a state where the microfoam is applicable in a medical intervention. The state that allows an application of the microfoam to a patient may require that the microfoam brings about a sclerosing effect and/or that the microfoam comprises uniform, tangent bubbles, preferably with a diameter of less than or at most 300 μm, more preferably less than 200 μm. The conservation process may stop after a specific time that may be fixed or selected by the user. It is further conceivable that the user may stop the conservation process, particularly via a command using the user interface.

In a fifth aspect, the present invention relates to a microfoam obtainable by a method according to the present invention.

In a preferred embodiment, the microfoam of the present invention is a “Kugelschaum” (spherical-bubble foam). The term “Kugelschaum” as used herein refers to a microfoam of spherical bubbles and are filled with the gas or gas mixture used for the foaming process.

In a further preferred embodiment, the microfoam of the present invention is a tangent bubble “Kugelschaum” wherein the bubbles are spherical and furthermore all bubbles have tangent neighbors. Tangent neighbors may in the context of the invention describe that the bubbles are touching each other in a single point, in particular without further interaction.

In another preferred embodiment the microfoam is an “Eigenschaum”. The term “Eigenschaum” as used herein refers to a Kugelschaum, wherein the individual bubbles are of essentially identical size thereby building a perfectly regular microfoam structure.

In a sixth aspect, the present invention relates to a microfoam which is essentially nitrogen-free. Accordingly, the microfoam has a nitrogen content of less than 2%, preferably less than 1% and more preferably less than 0.8%.

The microfoam according to feature combination 38 or 39, characterized in that the microfoam has a number of geometrical impurities being less than 30% of the bubble count, preferably less than impurities being less than 10% of the bubble count and more preferably less than 1% of the bubble count.

In the context of the present invention, a geometrical impurity is defined as a bubble that is 30% bigger or smaller than the mean radius of the sample, preferably 20% and most preferably 10%.

About the arrangement of the geometrical impurities the worst situation is when they are randomly scattered in the microfoam decreasing severely the half-life of the microfoam due to the onset of the Ostwald effect in multiple locations in parallel. Preferably the geometrical impurities should be in one of the sides of the sample, or in the top or in the bottom, more preferably all collected in a single block in the sides or in the top or in the bottom of the microfoam and more preferably in a spherical cluster in the centre of the microfoam that is produced in the foaming chamber.

The term “microfoam” as used herein refers to a foam with a mean bubble size of less than 100 μm, preferably less than 80 μm and more preferably less than 50 μm, measured 45 sec after foam preparation. As an example, the microfoam of the invention can have a mean bubble size of less than 95, 90, 85, 80, 75, 70, 65, 60 or 55 μm.

In one embodiment the microfoam of the present invention has a Sauter mean radius of less than 200 μm, preferably of less than 150 μm and more preferably of less than 100 μm, measured at 45 sec after the foam is prepared. As an example, the microfoam of the invention can have a Sauter mean radius of less than 190, 180, 175, 170, 165, 160, 155, 150, 145, 140, 135, 130, 125, 120, 115, 110, or less than 105 μm.

In a preferred embodiment, the microfoam has a moisture content of less than 20% (w/w), which is even more preferred between 8 and 12% of moisture content. As an example, the microfoam of the invention can have a moisture content of 9, 10, 11, 12, 13, 14, 15, 16, 17, 18 or 19%.

This microfoam can be prepared by the methods of the present invention.

In one particular example, the medical microfoam and/or the liquid composition may comprise an agent solution, such as polidocanol, in a liquid, such as water or physiological saline, at a concentration from 5 mg to 20 mg in 1 mL liquid (which corresponds to a concentration from 0.05 to 2.0% (w/v)).

In another example, the medical microfoam and/or in the liquid composition may comprise an agent solution, such as polidocanol, in a liquid, such as water or physiological saline, at a concentration from 2 mg to 5 mg in 1 mL liquid (which corresponds to 0.20-0.50% (w/v)).

In another particular example, the medical microfoam and/or in the liquid composition may comprise an agent solution of polidocanol in water or physiological saline at a concentration of 5 mg/mL.

In another particular example, the medical microfoam and/or in the liquid composition may comprise an agent solution of polidocanol in water or physiological saline at a concentration of 20 mg/mL.

In some embodiments, the microfoam of the invention may be used in diagnostic or therapeutic applications.

The diagnostic use includes the use of the microfoam as ultrasound contrast agent (USCA), whereby the intravenous injection of the microfoam can produce contrast enhancement in ultrasound images. The primary application of microfoam as USCA is in echocardiography, for ventricular opacification and delineation of endocardial borders. The microfoam can also be used for the assessment of systolic function and left ventricular volume, and for identifying myocardial infarction and coronary artery stenoses. The microfoam of the invention may be also suitable for the detection, characterisation and image guided treatment of focal liver lesions. Further applications include the functional investigations in other organs including the kidneys, spleen and pancreas and for assessment of disease in the bowel, prostate, breast and lymph nodes as well as non-vascular applications, such as assessment of fallopian tube patency and detection of ureteric reflux.

It is the highly non-linear response of the microfoam to ultrasound that has facilitated the development of a range of contrast-specific imaging protocols which have greatly increased the diagnostic potential of ultrasound imaging. For example, in combination with advances in three-dimensional visualisation techniques, harmonic imaging with microfoam has enabled mapping of the microcirculation for the characterisation of tumor vascularity, and also in the brain.

Microfoam can also be used in quantitative and targeted (molecular) imaging.

The ability to attach molecules to the microfoam-bubbles that are targeted to specific vascular receptor sites opens up further opportunities for tissue-specific imaging, and conditions such as inflammation, angiogenesis and atherosclerosis.

For use as USCA, it is preferred to use heavy gases that are less water soluble and therefore lead to a microfoam with an increased life span of the microfoam. Examples of heavy gases include sulfur hexafluoride, octafluoropropane, octafluorobutane, octafluoropentane, and octafluorohexane. Notably, there are currently four clinically available microfoam-based USCAs which are summarized in the following table:

| Name | Shell | Gas | Approved Indication |
|---|---|---|---|
| Sonovue ®/ | Phospholipid | Sulfur hexafluoride | left ventricular |
| limason ® | | | opacification |
| | | | endocardial border |
| | | | definition |
| | | | breast |
| | | | liver |
| | | | vascular |
| | | | urinary tract |
| Definity ®/ | Phospholipid | Octafluoropropane | left ventricular |
| luminity ® | | (Perflutren) | opacification |
| | | | endocardial border |
| | | | definition |
| | | | liver |
| | | | kidney |
| | | | vascular |
| Optison ® | Albumin | Octafluoropropane | left ventricular |
| | | (Perflutren) | opacification |
| | | | endocardial border |
| | | | definition |
| Sonazoid ® | Phospholipid | Octafluoropropane | breast |
| | | (Perflutren) | liver |
| | | | Myocardial perfusion |

In an alternative embodiment, the microfoam of the invention can be used for therapeutic applications. In drug and gene delivery, for example, the microfoam-bubbles can be used as targeted vehicles which are loaded with the required therapeutic agent, traced to the target site using low-intensity ultrasound and then, once attached, destroyed with a high-intensity burst to release the material locally, thus avoiding systemic administration, e.g., of toxic chemotherapy.

In gene delivery, the microfoam-bubbles can be used as targeted vehicles which are loaded with the required genetic material, which can be single or double stranded DNA or RNA, or which can be a gene vector such as a plasmid, a viral vector, a cosmid, or an artificial chromosome.

Sonodynamic therapy (SDT) is a more recent concept and involves the combination of ultrasound and a sonosensitising drug (also referred to herein as a “sonosensitiser”). In a manner similar to photodynamic therapy (PDT), activation of the sonosensitiser by acoustic energy results in the generation of reactive oxygen species (ROS), such as singlet oxygen, at the target site of interest. Such species are cytotoxic, thereby killing the target cells or at least diminishing their proliferative potential. Many known photosensitising agents can be activated by acoustic energy and are thus suitable for use in SDT. Since ultrasound readily propagates through several cm of tissue, SDT provides a means by which tumours which are located deep within the tissues may be treated. As with light, ultrasound energy can also be focused on a tumour mass in order to activate the sonosensitizer thereby restricting its effects to the target site.

SDT offers some significant advantages over PDT: ultrasound is widely accepted as a cost effective and safe clinical imaging modality and, unlike light, can be tightly focused with penetration in soft tissue up to several tens of centimetres depending on the ultrasound frequency used.

Accordingly, the gas-filled microbubble of the microfoam of the present invention can be conjugated to sonosensitisers to provide a microbubble-sonosensitiser “complex” for use in SDT. These complexes permit effective delivery of the active sonosensitiser in a site-specific manner by a controlled destruction of the bubble using ultrasound. Subsequent or simultaneous sono-activation of the targeted sonosensitiser results in cell destruction at the target site and regression of tumour tissues. The use of a microbubble also leads to a reduction in toxic side-effects due to the shielding of the sonosensitiser from potential light activation prior to reaching the desired target site.

It is known that motion of the microfoam-bubbles increases the permeability of both individual cell membranes and the endothelium including temporary opening of the blood-brain barrier. Accordingly, the bubbles of the microfoam of the invention can act not only as delivery vehicles, but also actively promote uptake of therapeutic material.

For a therapeutic microfoam a targeting can be also achieved by filling the microfoam bubbles with an acoustically active gas, that allows a radiation induced movement of the microfoam bubbles toward the target site.

In an alternative embodiment, the microfoam of the invention can be used for non-medical applications. Hereby, the microfoam of the present invention can also be used for cleaning purposes such as the cleaning of semiconductor wafers (see EP 2 202 782 B1).

In a seventh aspect, the present invention relates to a computer program comprising instructions for performing steps of the methods of the invention, which, when the program is executed by a computer, cause the computer to carry out the following steps:
a. Providing a user interface for controlling a foaming process for producing a microfoam, particularly by configuring at least one parameter of the foaming process and/or by initiating the foaming process, the at least one parameter being selected from the following: a gas concentration, a mixing ratio of the gas mixture, a selection of the liquid composition, a mixing ratio of the agent solution, a concentration of the agent solution in the microfoam, a language, a foaming profile (204),
b. providing at least one graphical element (201) as part of the user interface (200) for selecting or configuring the at least one parameter,
c. providing at least one graphical control element (202) for directing the computer program, in particular a graphical control element (202) for cancelling the computer program and/or a graphical control element (202) for continuing the computer program and/or a graphical control element (202) for moving one step backwards in the computer program,
d. providing at least one graphical section (203) as part of the user interface (200) showing at least one instructional image (205) and/or at least one informational image (206) for guiding or informing a user.

The user interface may be at least partly provided as a graphical user interface, particularly on a graphical screen. Said graphical screen may be provided as a touch screen. The at least one graphical element and/or the at least one graphical control element may be selected via the graphical screen by touching the graphical screen directly or via an interface, for instance via a mouse or a keyboard or at least one mechanical button. The instructional image may show graphical instructions, for instance the motion that is necessary by the user, of how to prepare the foaming process or how and/or where to inject an foaming agent and/or how to clean the apparatus. The informational image may comprise a status information of the foaming process or an information about how one or more parameters were selected for the foaming process or an information about a current state of at least one parameter during the foaming process. The user interface may be provided as at least two separate views wherein each view may comprise at least one graphical element and/or at least one graphical control element and/or at least one graphical section comprising at least one instructional image and/or at least one informational image. Said elements may vary in their size and/or location, the graphical section may additionally vary regarding the provided content within each view.

It may be conceivable that the computer program further comprises instructions which, when The program is executed by a computer, cause the computer to carry out a logging of important events and status information in a log file. The logging may include at least one of the following points of information: main activity on the user interface, changes to the device configuration, details about the amounts of agent and consumables being used for foaming, identification of cartridges being used, number of foaming cycles and total in-use time of cartridges, identification of hardware and software versions, results of power on self tests and or malfunctions during operation.

It is further conceivable that the computer program further comprises the following step:
providing at least one default value for the at least one parameter.

A default value may in this context be understood as a value that is preselected and that is used for the foaming process or for the user interface in case the user wishes to either keep said default value or forgets to change it.

In some embodiments, the foaming profile (204) of the computer program comprises at least one of the following parameters:
profile name and description,
names of foaming agent, diluent, gasses,
list of compatible cartridges,
available selection of agent input concentration,
valid range of agent input concentration,
volume of agent to be injected into the cartridge,
available selection of agent concentration in foam,
available selection of gas ratios,
detailed instructions of how to prepare the microfoam,
limit of keeping the microfoam fresh after preparation,
time limit for use of cartridge.
Foaming parameters such as rotation speed or ramp definitions

### Definitions

In the context of the invention, indefinite and definite articles or numerical indications, e.g. “one”, “two”, etc., are always to be understood as “at least” indications, unless expressly stated otherwise. Furthermore, numerical indications as well as indications of process parameters and/or device parameters are to be understood in the technical sense, i.e. as having the usual tolerances. Also, from the explicit indication of “at least” or the like it must not be concluded that by the simple use of the article or the numerical indication, i.e., without the indication of “at least” or the like, a restriction, e.g., in the sense of “exactly one”, is to be implied.

The phrase “in some embodiments” can be used interchangeably with “in one or more embodiments.

The gas or gas mixture as used herein may also be an “acoustically active gas” or mixture thereof. An “acoustically active gas” as used herein, refers to any gas (i.e., for example perfluorobutane) that may be entrapped within a bubble of the microfoam that vibrates in the presence of an acoustically generated radiation force, thereby inducing movement in the direction of the force. Suitable gases are inert and biocompatible, and include, for example, air, noble gases, such as helium, rubidium, hyperpolarized xenon, hyperpolarized argon, hyperpolarized helium, neon, argon, xenon, carbon dioxide, nitrogen, fluorine, oxygen, sulfur-based gases, such as sulfur hexafluoride and sulfur tetrafluoride, and fluorinated gases. The resulting microfoam is especially suited for diagnostic and therapeutic applications.

The term “fluid conduit” describes in the scope of the invention a conduit for transporting a fluid from one end, e.g., one part of the apparatus, to another, preferentially without loss of fluid, particularly preferred by providing a water-tight and/or gas-tight surrounding or path. The fluid conduit can be a tube, a channel or a pipe. Preferably, the fluid conduit is a flexible tube.

The term “liquid composition” may refer to a composition in liquid form. The liquid composition may include at least one foaming agent and may also refer to a liquid sclerosing composition. Using a foaming process, the microfoam may be produced from the liquid sclerosing composition and can therefore also be referred to as a foamed sclerosing composition. For example, using the foaming process the liquid composition is foamed by the rotation of the foaming element. The liquid composition may therefore form an ingredient to obtain the foamed sclerosing composition. The liquid compositions according to examples of the present disclosure comprise a foaming agent and also a suitable aqueous diluent as vehicle which can be injected without toxicity into the affected veins. In some examples, a liquid can be selected from sterile water (particularly distilled water) and physiological saline. This said liquid, which serves as the vehicle, may in the scope of the present invention be referred to as the diluent.

In other words, and according to a first aspect of the present inventive concept, the object is solved by a Cartridge for housing the producing of a therapeutical microfoam, comprising
a. a housing body,
   the housing body comprising a hollow interior; and
b. a reaction unit within the hollow interior,
   the reaction unit comprising a reaction unit body,
   the reaction unit body comprising a foaming chamber; and
c. a foam extraction port
   arranged accessible from an outside of the housing body for extracting produced microfoam.

Considering several options for the foam extraction the Cartridge according to claim 1 can be such that the foam extraction port is located at a surface of the housing body, preferably at a top or front surface of the housing body.

The housing body can comprise, within the hollow interior, a foam extraction conduit fluidically coupling the reaction unit, preferably the foaming chamber, to the foam extraction port.

The foam extraction conduit can at least partly be constructed as a part of the reaction unit body.

The foam extraction conduit can comprise a fluidic connection to a foam extraction opening of the foaming chamber, the foam extraction opening being located in a lower half of the foaming chamber, preferably reaching a bottom of the foaming chamber.

The foam extraction conduit optionally connects a channel outlet in the reaction unit body with the foam extraction port.

The foam extraction conduit preferably comprises a valve.

The foam extraction conduit might exclusively consist of rigid parts, free of hosing, the rigid parts preferably consisting of a polymer or monomer plastic material.

The foam extraction port or a foam extraction conduit might comprise a foam extraction filter.

The foam extraction port might comprise a detachable foam extraction port sealing member and/or a foam extraction conduit valve.

The foam extraction port might comprise a foam extraction reservoir, so that the foam that has been created in the foaming chamber might be intermediately stored before it is extracted.

The foam extraction port might comprise a syringe dock. This might be a Luer taper or a Luer lock, or a bayonet lock, or a rubber-based lock, or any further options.

As to proposed option for the gas supply, the cartridge might comprise a gas supply port. In case there is a foam extraction port, the gas supply port might preferably be different from the foam extraction port.

The gas supply port might be located at a surface of the housing body, preferably at a bottom or back surface of the housing body.

The housing body might comprise, within the hollow interior, a gas supply conduit, fluidically coupling the gas supply port to the reaction unit, preferably to the foaming chamber.

The gas supply conduit might comprise a gas supply opening of the foaming chamber, the gas supply opening being located in an upper half of the foaming chamber, preferably on a top lid of the foaming chamber.

The gas supply conduit might comprise a hose, preferably a translucent hose.

The gas supply conduit might comprise a pressure relieve, especially opening above a pressure of 80+/−20 or preferable +/−10 mbar.

The gas supply port or a gas supply conduit might comprise a gas supply filter.

The gas supply port might comprise a detachable gas supply port sealing member. This way, the interior of the cartridge can be safely sterile.

The cartridge might comprise exactly one gas supply port.

The cartridge might, as an alternative, comprise two gas supply ports, and wherein within the interior of the housing body, there is a merging junction for gas coming from the two gas supply ports.

As far as a saline liquid supply is concerned, the cartridge might further comprise a diluent supply port, which is in case of claims 2 to 12 different from the foam extraction port and/or which is in case of claims 13 to 22 different from the gas supply port.

The diluent supply port might be located at a surface of the housing body, preferable at a bottom or back surface of the housing body, so that it would easily connect to fluid connecting elements on a foam preparation device.

The housing body might comprise, within the hollow interior, a diluent supply conduit, fluidically coupling the diluent supply port to the reaction unit, preferably to the foaming chamber.

The diluent supply conduit might comprise a diluent supply opening being located in an upper half of the foaming chamber, preferably on a top lid of the foaming chamber.

The saline supply conduit might comprise a hose, preferably a translucent hose.

By providing a hose, the hollow interior space can be used in many variants. Especially, the length of the hose can be designed quite freely in order to meet the constructive needs.

The saline supply conduit hose Is preferably longer than a gas supply conduit hose.

By leading the liquid saline within the hollow interior of the cartridge, it is possible to lead the saline e.g. to a sensor unit.

The saline supply port or a saline supply conduit might comprise a saline supply filter.

The saline supply port might comprise a detachable saline supply port sealing member.

As far as options for a foaming agent supply are to be concerned, the cartridge might further comprise a foaming agent supply port, which is in case of claims 2 to 12 different from the foam extraction port and/or which is in case of claims 13 to 22 different from the gas supply port and/or which is in case of claims 23 to 30 different from the saline supply port.

The foaming agent supply port might be located at a surface of the housing body, preferably at a top or front surface of the housing body and/or on the same side of the housing body as the foam extraction port.

This way, the practitioner can reach the foaming agent supply port easily, and can add, preferably inject, most preferably inject with a syringe needle, foaming agent liquid into the cartridge for in situ production of microfoam.

The housing body might comprise, within the hollow interior, a foaming agent supply conduit, fluidically coupling the foaming agent supply port to the reaction unit, preferably to the foaming chamber.

The foaming agent supply conduit might comprise a foaming agent supply opening being located in an upper half of the foaming chamber, preferably on a top lid of the foaming chamber.

The foaming agent supply port might comprise an injection port for receiving a syringe needle.

The housing body might comprise, in its hollow interior, a foaming agent reservoir which is fluidically coupled but apart from the foaming agent port and/or which is fluidically coupled but apart from the foaming chamber.

The foaming agent reservoir might be included in a foaming agent supply conduit for receiving and forwarding the foaming agent in a unidirectional flow path from the foaming agent supply port to the foaming chamber.

The foaming agent reservoir might be fluidically connected to the foaming agent supply conduit for receiving and forwarding the foaming agent in a bidirectional flow path from the foaming agent supply port to the foaming chamber.

The foaming agent reservoir might be a hollow body with an ejector.

Having a hollow body, the reservoir provides a space for receiving and intermediately holding the foaming agent, before the foaming agent is forwarded, full or in part, to the foaming mechanism, especially into the foaming chamber.

Having an ejector, the ejector can exert the pressure needed for forwarding the foaming agent.

The ejector can be controlled, so that it becomes possible to forward a predetermined/calculated amount of the foaming agent, e.g. a part of the foaming agent.

The ejector might be a passive element which is movable by an ejection actor which is located outside the cartridge, preferable the housing body comprising an ejector access opening in its surface, wherein the foaming agent reservoir might be a syringe.

As an alternative or as an addition, the ejector might be a passive element which is movable by an ejection actor which is located within the hollow interior of the housing body.

The ejection actor might be a pneumatic drive.

The ejection actor might be an electric drive.

The cartridge might comprise a valve for selectively opening or closing and/or redirecting a fludical path from the foaming agent supply port to the foaming chamber and/or to the foaming agent reservoir.

The cartridge might comprise a valve for selectively opening or closing and/or redirecting a fludical path from the foaming agent reservoir to the foaming chamber.

The foaming agent supply conduit might comprise a T-junction tube element.

The foaming agent supply conduit might comprise a foaming agent filter.

The cartridge might comprise a detachable foaming agent port sealing member.

As far as options for the reaction unit and a waste chamber and the foaming chamber are concerned, the Cartridge might be such that the cartridge further comprises, within the reaction unit for producing a microfoam, within the foaming chamber or fluidically connected to the foaming chamber, a foaming element.

The foaming element might comprise a pressure-filter unit, wherein a filter is provided at a pressure relief so that a sclerosant composition pressed under pressure through the filter would create a foam.

The foaming chamber might comprise a rotatable foaming element arranged in the foaming chamber for performing a foaming process to produce the microfoam from a liquid foaming composition when driven rotatably and/or for performing a foam maintaining process to maintain a created foam when driven rotatably- or in order to maintain a foam that has been created using either the rotatable foaming element for foaming, or a different foaming means, or both.

In practice, a rotatable element will make sense at least to be rotated for keeping the foam in a condition usable for the practitioner for a certain period of time, e.g. for a time window of approx. three to seven minutes, e.g. around five minutes.

The foaming chamber might comprise a positioning member on the bottom of the foaming chamber that holds the foaming element to be rotatable around a predefined axis during the foaming process,

The foaming chamber might comprise a first outlet channel, namely a foam extraction opening, provided adjacent to or at the bottom part of the foaming chamber that is in fluid communication with the said foaming chamber for removing the microfoam from the foaming chamber.

The reaction unit defining the foaming chamber further might have one or more of the following characteristics, and preferably all of the following characteristics:
a first inlet channel, namely a gas supply opening, that is in fluidical communication with the said foaming chamber for supplying a gas or gas mixture to the foaming chamber, whereby the first inlet channel is preferably provided at the top part of the foaming chamber; and/or
a second inlet channel, namely a foaming agent supply opening and/or a diluent supply opening or a combined foaming agent/diluent supply opening, that is in fluid communication with the said foaming chamber for supplying a mixture comprising a foaming agent and an aqueous diluent to the foaming chamber, whereby the second inlet channel is preferably provided adjacent to or at the bottom part of the foaming chamber, or
a second inlet channel, namely a foaming agent supply opening and/or a diluent supply opening or a combined foaming agent/diluent supply opening, that is in fluid communication with the said foaming chamber for supplying an aqueous diluent to the foaming chamber, whereby the second inlet channel is preferably provided at the top part of the foaming chamber, provided together with a third inlet channel, namely a foaming agent supply opening, that is in fluid communication with the said foaming chamber for supplying a foaming agent to the foaming chamber, whereby the third inlet channel is preferably provided adjacent to or at the bottom part of the foaming chamber.

The reaction unit defining the foaming chamber further might have a second outlet channel, namely a waste outlet opening, that is in fluid communication with the said foaming chamber for removing waste material from the foaming chamber whereby the second outlet channel is preferably provided adjacent to or at the bottom part of the foaming chamber.

The reaction unit body defining the foaming chamber further might have a deflection element which comprises a surface which reduces a free axial-parallel height within the foaming chamber along a circumferential path, so that if a fluid, especially a foam, is rotated within the foaming chamber along a circumferential path, part of the foam is subjected to a forced deflection generating a downward fluid motion during the foaming process in direction to the rotating foaming element at the bottom part.

The reaction unit body preferably is a sealed reaction unit body and/or a sterile or sterilizable reaction unit body.

The rotatable foaming element, if the choice has fallen on a rotatable foaming element, might configured as a disc with mixing element protruding radially and located around an outer perimeter of said disc, whereas the mixing features comprise one or more of the following: blades, rings, teeth, fins, rips, wings, arms, fingers, perforated plates, grid structures, or a ring-shaped helical spring.

The rotatable foaming element, being preferably a disc, might be configured to be magnetically driven by a magnetic stirrer located outside the foaming chamber and preferably beneath the bottom part of the foaming chamber, and comprises a magnetically couplable element.

The rotatable foaming element might have a centrally located bore, which is preferably open at one end for receiving the positioning member.

The positioning member might be a centrally or eccentrically located shaft on the bottom of the foaming chamber and preferably has a cylindrical side wall with a rounded upper corner.

The reaction vessel might further comprise a restriction member for restricting an upward movement of the rotatable foaming element, whereby said restriction member is preferably a rod-shaped element extending from the top part of the foaming chamber.

The deflection element might be a deflector plate or deflector body, or a pin-like structure, located in the foaming chamber and/or a propellor provided on the rotating member.

The deflector body might be a circular ring segment of trapezoidal basic structure with a beveled side towards the bottom of the foaming chamber and a bottom horizontal side situated above the rotatable foaming element to provide a gap between the deflector body and the upper margin of the rotatable foaming element, whereby the circular ring segment is preferably attached to the cylindrical wall of the foaming chamber, having more preferably a radial ring thickness which covers the mixing features of the rotatable disc.

The fluid dynamical effect of the deflector body would be to reduce the free height of the circumferential current, so as to induce a secondary current into the circumferential current. This leads to an enhanced mixing result for the foam in the foaming chamber, leading to an enhanced uniformity of the foam properties.

The channel providing the free height for the circumferential current might be situated under the bottom side, ideally under the bottom horizontal side, of the deflector body.

The reaction unit body should contain a gas distributor system, preferably selected from:
a deviation plate or body located in the foaming chamber beneath the first inlet channel to induce a lateral deflection of the gas stream entering the foaming chamber, or
a semispherical-, conical-, frustoconical- or dome-shaped top part of the foaming chamber with the first inlet channel at the top enabling a gas flow which is uniformly distributed over the horizontal cross-section of the foaming chamber.

The surface of an upper section of the foaming chamber might be coated with a hydrophobic or superhydrophobic agent or is a morphologically structured (super) hydrophobic surface such as a superamphiphobic, a slippery liquid-infused porous surface (SLIPS), or a liquid-impregnated surface (LIS).

The reaction unit further might further comprise
d. a waste container defining a waste chamber containing an inlet for receiving the waste material from the foaming chamber, and
e. a fluid conduit coupling the second outlet channel of the foaming chamber through a valve with the said inlet of the waste container, and
f. the waste container might preferably comprise an outlet for pressure release.

The inlet might be positioned in the top part of the waste container, preferably as the upper aperture of a hollow shaft positioned preferably in the center of the waste chamber.

If any foam and fluid leftovers are spilled into the waste chamber from above the current waste chamber surface level, a production of additional bubbles can be minimized or avoided, so that the available space in the waste chamber can be used best.

The valve for controlling the flow of the waste material into the waste chamber might be located on the outer surface or accessible from an outer surface of the cartridge and can be controlled by exerting an external pressure on said valve.

The inner surface of the waste chamber might be coated with a defoaming agent or has a morphologically structured defoaming inner surface such as an anti-foaming surface such as a superamphiphobic, a slippery liquid-infused porous surface (SLIPS), or a liquid-impregnated surface (LIS).

This would further help decomposing foam, thereby best using the available space of the waste chamber.

A waste chamber volume might be larger than a foaming chamber volume, preferably the waste chamber volume being from >=1 to <=3 times the foaming chamber volume.

The waste chamber is of particular importance when the cartridge is to be used for several foaming production processes in a row, e.g. for two or three amounts of foaming for the treatment of one single patient. The leftovers in the foaming chamber can then ideally be rinsed into the waste chamber, so that for the consecutive foaming process, a pre-predictable condition within the foaming chamber can be secured.

The waste chamber and the foaming chamber might be adjacent to each other, having a joint wall.

This way, the reaction unit body can be a two-chambers body, one chamber for the foaming chamber, and one chamber for the waste chamber.

A fluidic connection between the foaming chamber and the waste chamber can connect to the foaming chamber at a bottom of the foaming chamber and open into the waste chamber at a top region of the waste chamber.

As far as advantageous options for the cartridge as such are concerned, the cartridge might further comprise, on its exterior, a fully closed surface, comprising, preferably consisting of, the housing body and multiple sealing members.

The sealing members can be detachable glued tapes.

The cartridge can comprise, preferably within the hollow interior, an electronically readable identifier means, preferably selected from an RFID tag, a barcode, a data matrix code, a QR code or a different optical code.

This way, and with the coupling of a reading means in the automated device, the device can secure that only genuine cartridges can be used within the device.

If the device comprises a reader for an electronically identifier means at or preferably in the cartridge, the device might ideally be programmed to at least record in a uses protocol which cartridge was used, and preferably not activate the GUI start options for the foam production in case a cartridge is inserted which is either not quality-controlled by the original manufacturer, or which was e.g. used before.

According to a second aspect of the invention concept, the object is solved by an automated device for generating a therapeutical microfoam comprising a cartridge holder adapted to hold a replaceable cartridge according the specification in the present patent application, especially according to any of claims 1 thru 76.

The device might comprise a magnetic stirrer actor situated beneath the cartridge holder, for generating a rotating magnetic field for controlling the rotation of the foaming element around the positioning member upon inserting of a cartridge.

The cartridge holder might preferably contain a gas feed port that is configured to fluidly engage with the gas supply port of a cartridge upon insertion in the holder, thereby providing a gas-tight seal of both ports, wherein the gas feed port or the system preferably contains a safety valve allowing a gas supply only for a cartridge being properly locked in the cartridge holder.

The cartridge holder might, preferably in addition, contain a diluent feed port that is configured to fluidly engage with the diluent supply port of the cartridge in the holder, thereby providing a fluid-tight seal of both ports, wherein the diluent feed port or the system preferably contains a safety valve allowing a diluent supply only for a cartridge being properly locked in the cartridge holder.

The automated device might contain a sensor that is configured to detect within the cartridge the presence or flow of a liquid in the fluid conduit coupling the diluent port and the second inlet channel of the foaming chamber, whereby said sensor is preferably a capacitive sensor or an ultrasonic sensor.

The cartridge holder might contain one or more guiding elements for restricting an insertion of the cartridge within the cartridge holder to a zero tolerance seat, wherein the guiding device preferably contains an, preferably at least two, retractable members arranged at the bottom of the cartridge holder and mate with respective orifices of the cartridge housing.

The cartridge holder might contain a fastening mechanism configured to fasten the cartridge to the cartridge holder, whereby the fastening mechanism preferably contains one or more members that are arranged in the lateral wall of the cartridge holder and can be extended to engage with respective features of the cartridge housing.

The automated device might contain a gas supply system containing:
at least one gas port for connection to at least one gas container, being preferably a gas bottle,
at least one gas conduit connecting the at least one gas port to the gas supply port via a valve for regulating the gas flow,
optionally a pressure compensation device for compensating for pressure transients in the pressure within the foaming chamber.

The automated device contains a gas supply system might, in a preferred embodiment, comprise
two gas ports for connection to two gas bottles containing different gases, preferably installed in the automated device and being more preferably an O₂ gas bottle and a CO₂ gas bottle,
a gas channel network between the two gas ports and the gas feed port,
a gas mixing device for mixing the two different gases in a selectable ratio, and
optionally a pressure compensation device for compensating for pressure transients in the pressure within the foaming chamber.

The automated device might, preferably in addition, contain a diluent supply system containing
a diluent port for connection to a diluent container, preferably installed in the automated device, and
a liquid conduit connecting the diluent port to the diluent feed port via a pump capable of transporting the diluent from the diluent container to the diluent feed port.

The automated device might comprise a syringe-associated delivery system containing a drive unit moving an agitator which can perform at least one of the following actions:
detecting the correct connection of the inserted syringe to the foaming agent reservoir port of the said holding member; and/or
detecting the complete filling of the syringe via the foaming agent reservoir port by detecting the final position of the plunger; and/or analoguously if a non-syringe based reservoir is used; and/or
delivering a determined amount of foaming agent to the foaming chamber by pushing the plunger of the foaming agent-containing syringe to a controlled extent,
optionally detecting malfunctions of the syringe by determining the amount of pressure to be executed on the plunger of the syringe.

The system might further comprise:
a control computing unit, in which a control program is located connected with a user interface for controlling the foaming process, particularly by configuring at least one parameter of the foaming process and/or by initiating the foaming process, the at least one parameter being directly or indirectly selected from the following:
   i. a gas concentration,
   ii. a mixing ratio of the gas mixture,
   iii. a concentration of the foaming agent in the mixture of foaming agent and diluent,
   iv. the total amount of the mixture of foaming agent and diluent, selection of the liquid composition,
   v. the rotation speed and time of the rotatable foaming element,
   vi. the removal of waste material from the foaming chamber,
   vii. the cleansing of the foaming chamber as preparation for a further foaming process,
   viii. the removal of the cartridge from the cartridge holder,
wherein the automated device is configured to perform the foaming process based on the configured parameter automatically, and particularly the mixing of the gases according to the configured mixing ratio of the gas mixture automatically.

There might be a conjunction of two gas supply conduits external of the cartridge, so as to unite a gas supply of two gas sources located within the device into one common fluidical stream into the cartridge.

The cartridge holder might comprise a replaceably inserted and connected cartridge according to any of claims 1 to 77.

As soon as the device is electrically powered, where preferably the device has to be powered in order to accept the insertion and locking of a cartridge.

A computer program might comprise instructions for performing steps which, when the program is executed by a computer, cause the device to carry out the following steps:
Providing a graphical user interface (GUI) for controlling a foaming process for producing a microfoam, particularly by configuring at least one parameter of the foaming process and/or by initiating the foaming process, the at least one parameter being selected from: a gas concentration, a mixing ratio of the gas mixture, a selection of the liquid composition, a mixing ratio of the agent solution, a concentration of the agent solution in the microfoam, a foaming profile (204);
providing at least one graphical element (201), touch-controlled or physical, as part of the user interface (200) for selecting or configuring the at least one parameter;
providing at least one graphical control element (202) for directing the computer program, in particular a graphical control element (202) for cancelling the computer program and/or a graphical control element (202) for continuing the computer program and/or a graphical control element (202) for moving one step backwards in the computer program and/or a graphical control element for rinsing the foaming chamber and producing another amount of foam without exchanging the cartridge;
providing at least one graphical section (203) as part of the user interface (200) showing at least one instructional image (205) and/or at least one informational image (206) for guiding or informing a user.

The foaming profile (204) might comprise at least one of the following parameters:
profile name and description,
names of foaming agent, diluent, gasses,
list of compatible cartridges,
available selection of agent input concentration,
valid range of agent input concentration,
volume of agent to be injected into the cartridge,
available selection of agent concentration in foam,
available selection of gas ratios,
detailed instructions of how to prepare the microfoam,
limit of keeping the microfoam fresh after preparation,
time limit for use of cartridge.

A timer might be comprised which defines a maximum time for using a prepared microfoam, preferably signalling when the maximum time has been used up.

Ideally, when the designated time has run out, the device can be configured to initiate a rinsing of the foaming chamber and/or of the foam extraction conduit.

According to a third aspect of the inventive concept, the object is solved by a method for automatically producing a therapeutic microfoam comprising the following steps:
a. providing an automated device according to the description in the present patent application, especially any of patent claims 78 to 95;
b. inserting a cartridge according to the description in the present patent application, especially according to any of patent claims 1 to 77 into the cartridge holder of the automated device;
c. selecting a foaming agent concentration using a user interface,
d. optionally selecting a ratio for the mixture of two gases using a user interface,
e. providing, preferably injecting, a foaming agent, preferably through a sealing member, of a foaming agent supply port,
f. starting a foam generation process using a user interface; until a suitable microfoam has been obtained in the reaction unit,
g. once the foam has been obtained, extracting microfoam through a foam extraction port of the cartridge.

Additional steps might be of advantage:
optionally flushing the foaming chamber with a selected gas or gas mixture or rinsing with the aqueous diluent, being preferably saline; and/or
introducing the selected gas or gas mixture into the foaming chamber via the first inlet channel; and/or
introducing a mixture of the foaming agent and the aqueous diluent into the foaming chamber via the second inlet channel, or alternatively, introducing the aqueous diluent into the foaming chamber via the second inlet channel and the foaming agent into the foaming chamber via the third inlet channel; and/or
initiating a rinsing process in the foaming chamber by using a user interface; and/or
repeating the process according steps f and g, preferably e, f and g, or d, e, f and g, thereby maintaining the same cartridge; or
exchanging the cartridge by removing the used cartridge and inserting a new, sealed cartridge.

The microfoam which is obtained is of superior quality for the designated therapeutic use: The therapeutic microfoam, preferably obtained by a method according to any of claims 96 to 99 and/or using a cartridge according to any of claims 1 to 77 and/or using a device according to any of claims 78 to 95, has, according to numerous test performed by the inventors, can be identified by one or more of the following properties, which are absolutely unique in this technical field:
a. the microfoam has a nitrogen content of less than 2%, preferably of less than 1% and more preferably of less than 0.8%; and/or
b. the microfoam has a number of geometrical impurities being less than 30% of the bubble count, preferably less than impurities being less than 10% of the bubble count and more preferably less than 1% of the bubble count; and/or
c. the microfoam has a mean bubble size of less than 100 μm, preferably less than 80 μm, and more preferably less than 50 μm, measured 45 seconds after foam preparation; and/or
d. the microfoam has a Sauter mean radius of less of less than 200 μm, preferably less than 150 μm, and more preferably less than 100 μm, measured 45 seconds after foam preparation; and/or
e. the microfoam has a moisture content of less of less than 20% (w/w), being preferably between 8 and 12% (w/w).

The use of the microfoam according to the above-identified features, especially according to patent claim 100, and/or of a microfoam obtained by a method according to any of patent claims 96 to 99 and/or using a cartridge according to any of patent claims 1 to 77 and/or using a device according to any of patent claims 78 to 95, for the therapeutic treatment of a human patient's varicose vein, by injecting a sclerosant solution, in the form of the microfoam, into the affected vein, thereby displacing blood in the vein and leading to a closure of the affected vein, makes the inventive concept stand out over the prior art, and is of direct advantage for the patients and for the practitioners.

Further advantages, features and details of the invention will be apparent from the following description, in which embodiments of the invention are described in detail with reference to the drawings. In this connection, the features mentioned in the claims and in the feature combinations and in the description may each be essential to the invention individually or in any combination. Showing:

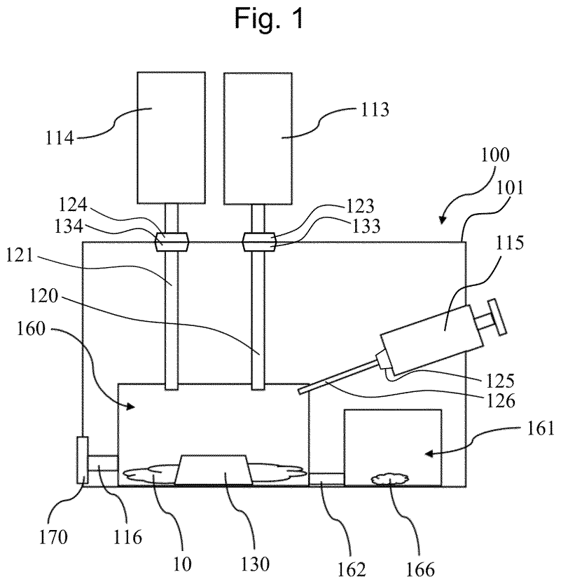

FIG. 1 in a schematic cross section an embodiment for a cartridge,

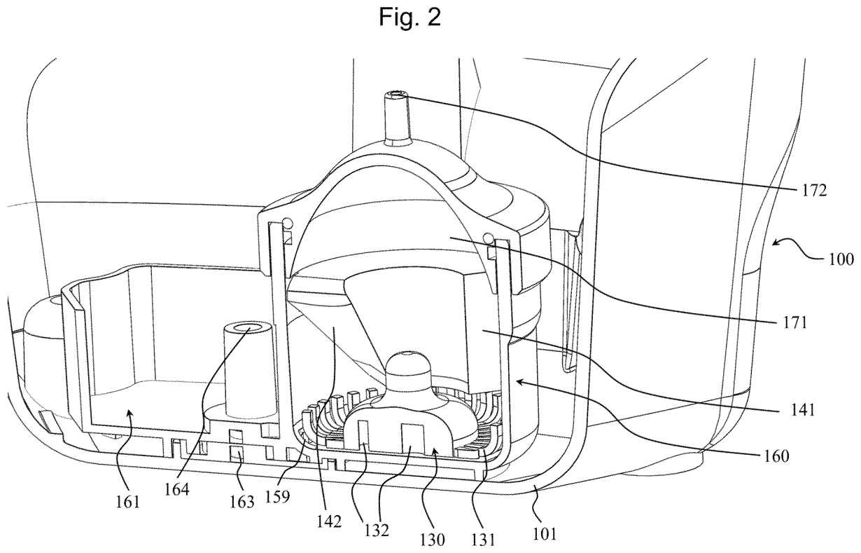

FIG. 2 Drawing of a cross section showing an embodiment for the cartridge of the invention, whereas the partial view depicts the reaction chamber and the waste chamber,

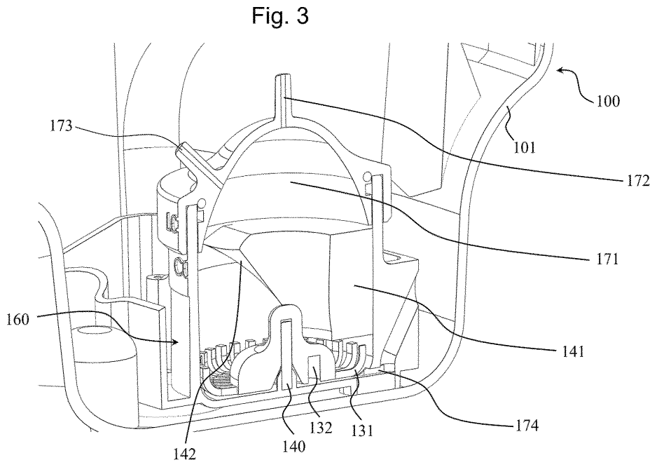

FIG. 3 Drawing of a cross section showing an embodiment for the reaction chamber of the invention, which is depicted as to be contained within the cartridge of the invention,

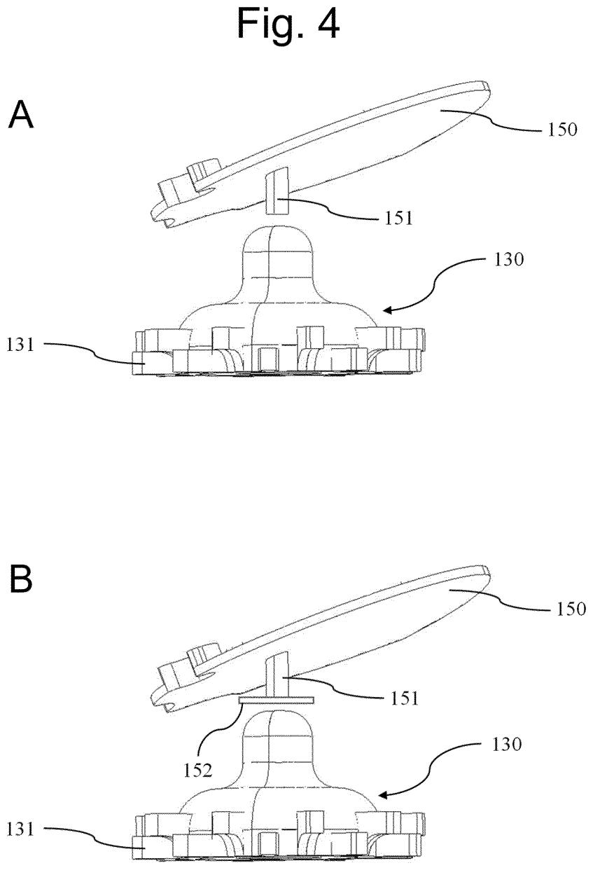

FIG. 4 Schematic drawing showing an example of an inventive foaming element and restriction arrangement with a protrusion (A) or with a stop plate (B),

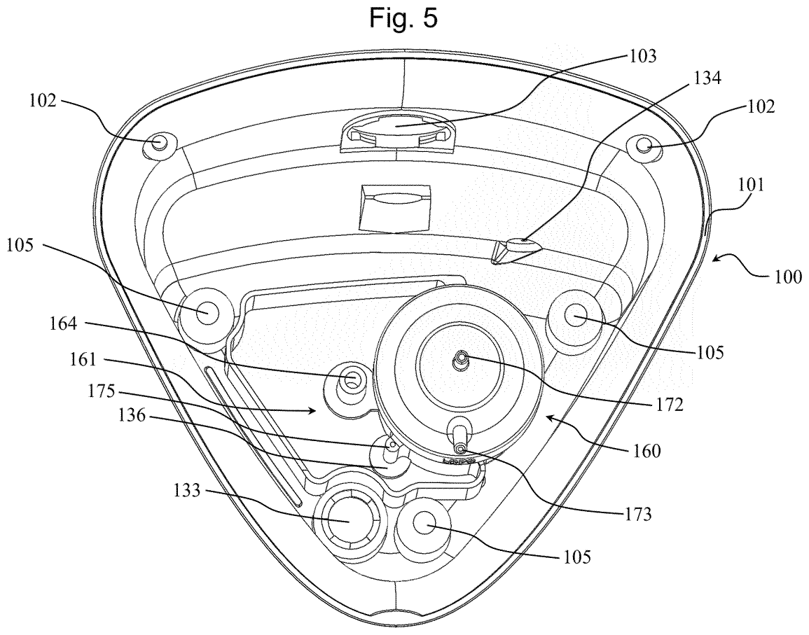

FIG. 5 Drawing showing an embodiment for the cartridge of the invention, which is viewed from slanted from above after removal of the top lid and removal of the fluid conduits,

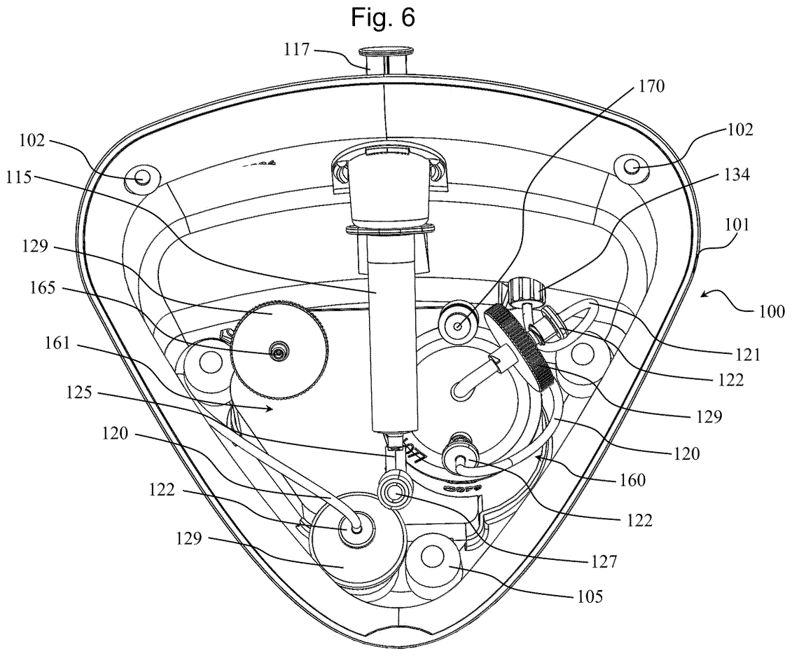

FIG. 6 Drawing showing an embodiment for the cartridge of the invention, which is viewed from slanted from above after removal of the top lid,

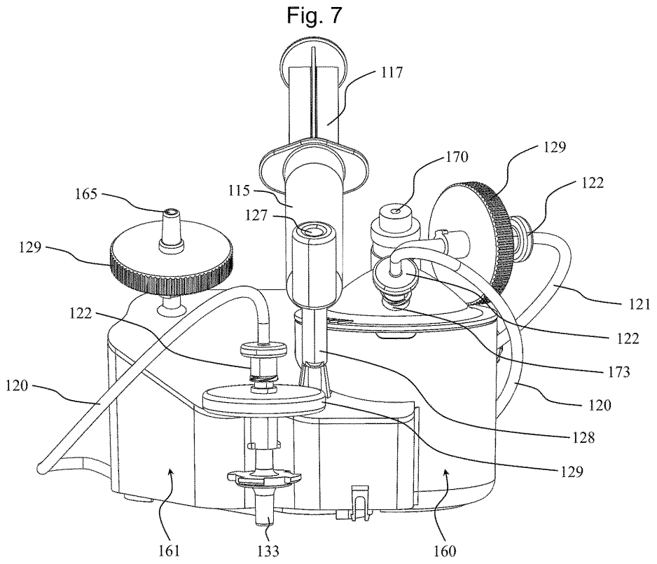

FIG. 7 Drawing showing an embodiment for the cartridge of the invention, which is viewed from slanted from above after removal of complete cartridge housing,

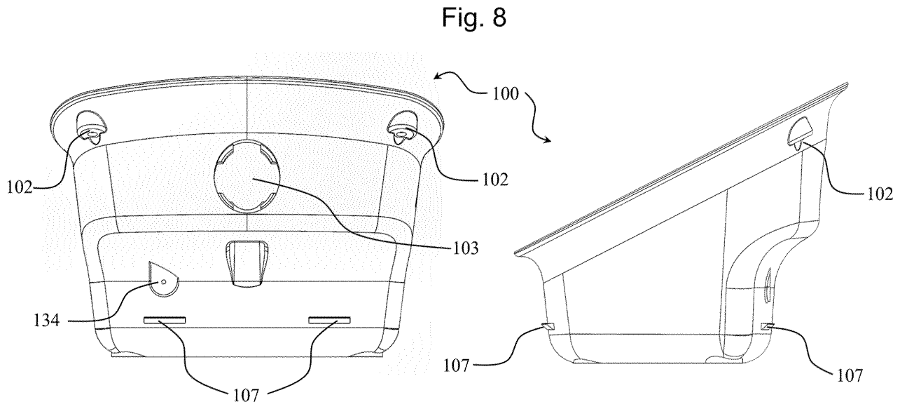

FIG. 8 Drawing showing an embodiment for the cartridge of the invention in back view and side view after removal of the syringe 115,

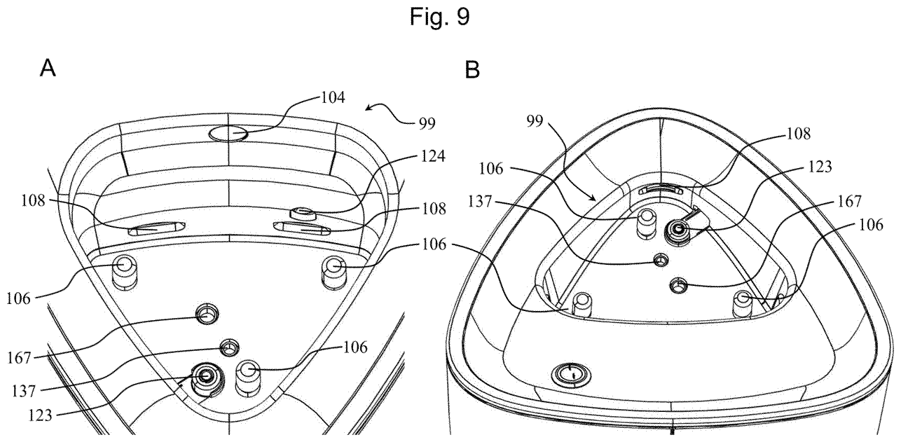

FIG. 9 Drawing showing an embodiment for the cartridge holder of the automated device of the invention in front view (A) and back view (B) both viewed at an angle from above without the cartridge being inserted,

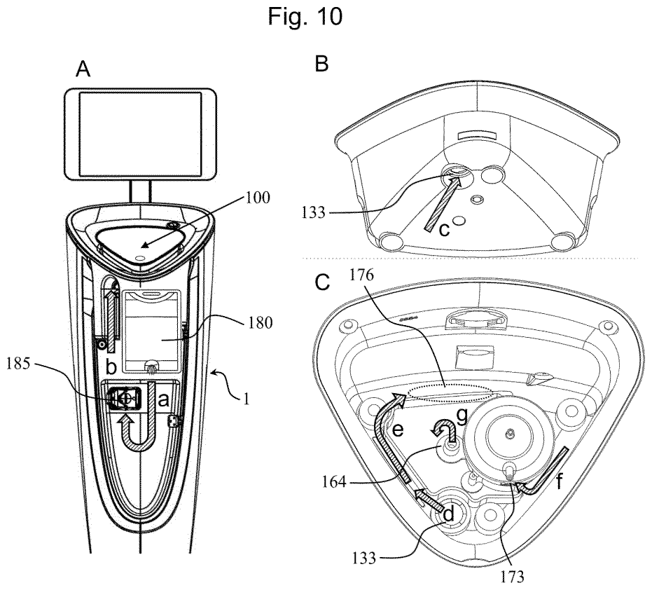

FIG. 10 Schematic drawing depicting the flow path for the aqueous diluent within the automated device 1 of the invention including the cartridge 100,

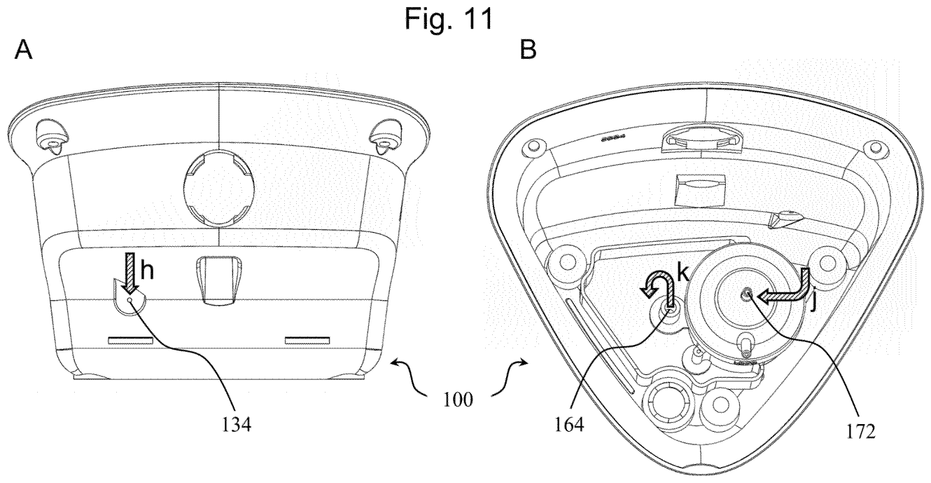

FIG. 11 Schematic drawing depicting the flow path for the gas within the cartridge 100 of the invention,

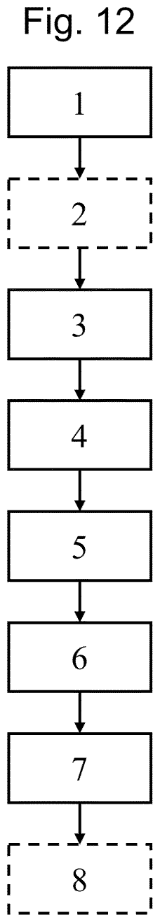

FIG. 12 Flow diagram depicting the process step for the microfoam generation using an automated device 1 and cartridge 100 according to one embodiment of the present invention,

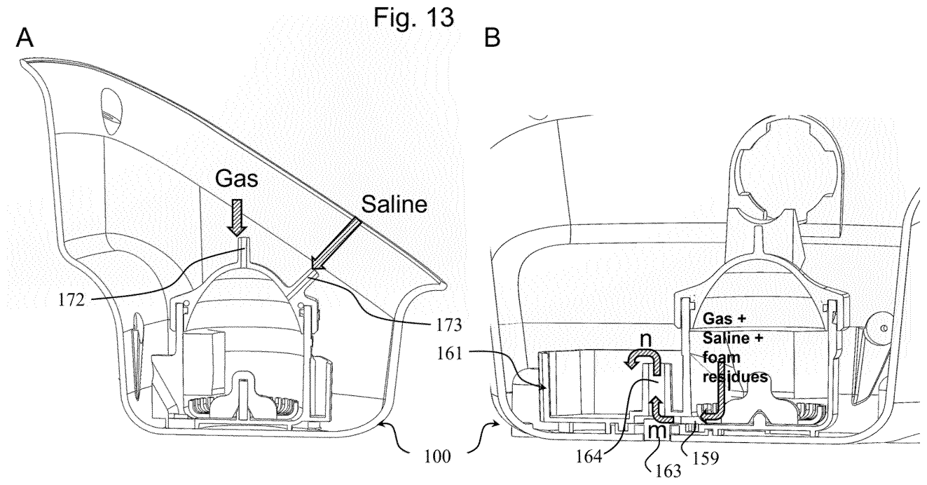

FIG. 13 Schematic drawing of cross sections of a cartridge according to one embodiment of the invention showing the cleaning steps for the foaming chamber,

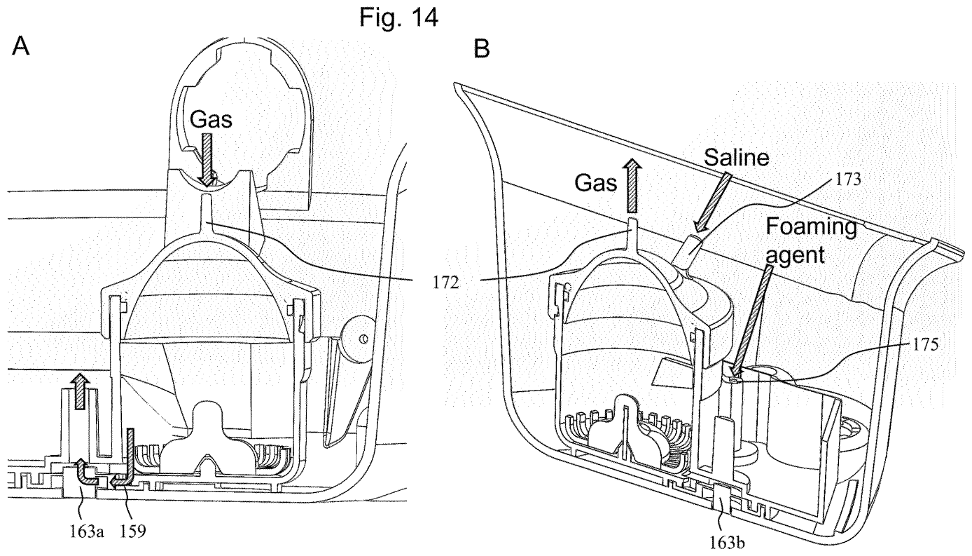

FIG. 14 Schematic drawing of cross sections of a cartridge according to one embodiment of the invention showing steps which prepare the foaming chamber for the foaming process,

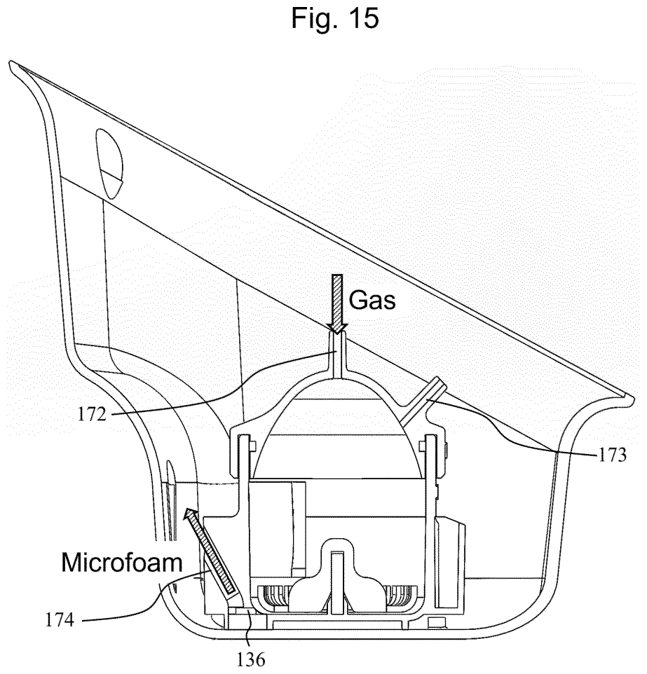

FIG. 15 Schematic drawing of a cross section of a cartridge according to one embodiment of the invention showing the step of microfoam removal from the foaming chamber,

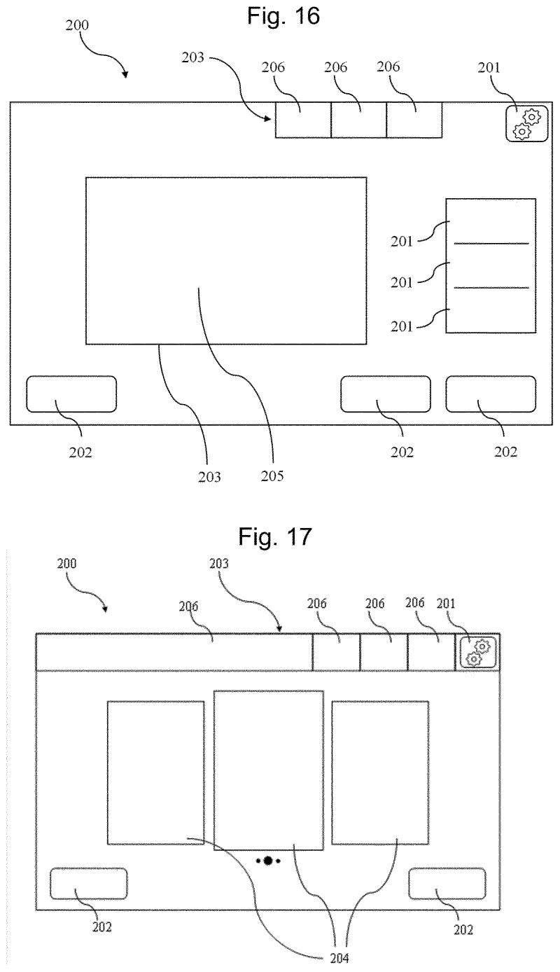

FIG. 16 Conceptual view of an inventive user interface,

FIG. 17 Conceptual view of an inventive user interface wherein a foaming profile is to be selected.

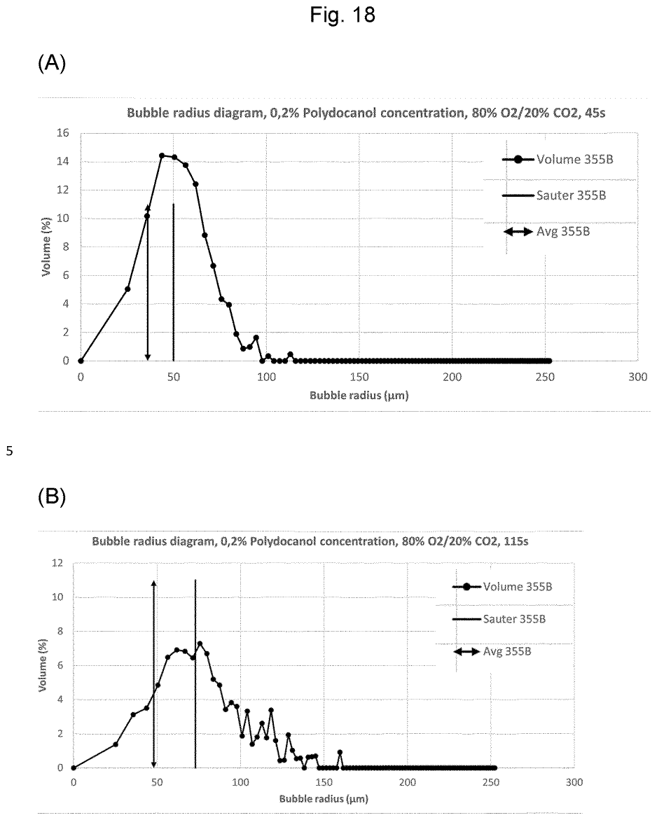

FIG. 18 The bubble distribution for a microfoam according to the invention. The measurements were recorded 45 sec (A) or 115 sec (B) after foam preparation of the same microfoam.

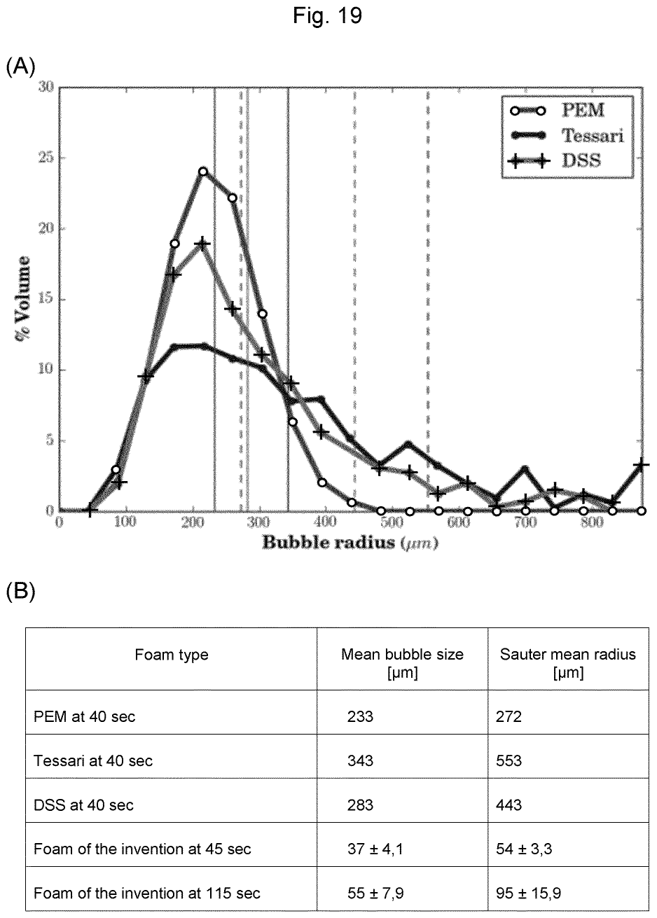

FIG. 19 (A) Bubble distribution for PEM, Tessari and DSS samples as shown in FIG. 3 of Roberts et al, 2020 (Biorheology; 57 (2-4): 77-85); (B) Table depicting measurements extracted from the data for PEM, Tessari and DSS (see Tables 1 to 3 of Roberts et al. 2000), in comparison with measurements extracted from the data of the microfoam of the invention as shown in FIGS. 18A and 18 B. The table lists the mean bubble size and the Sauter mean radius, both given in μm.

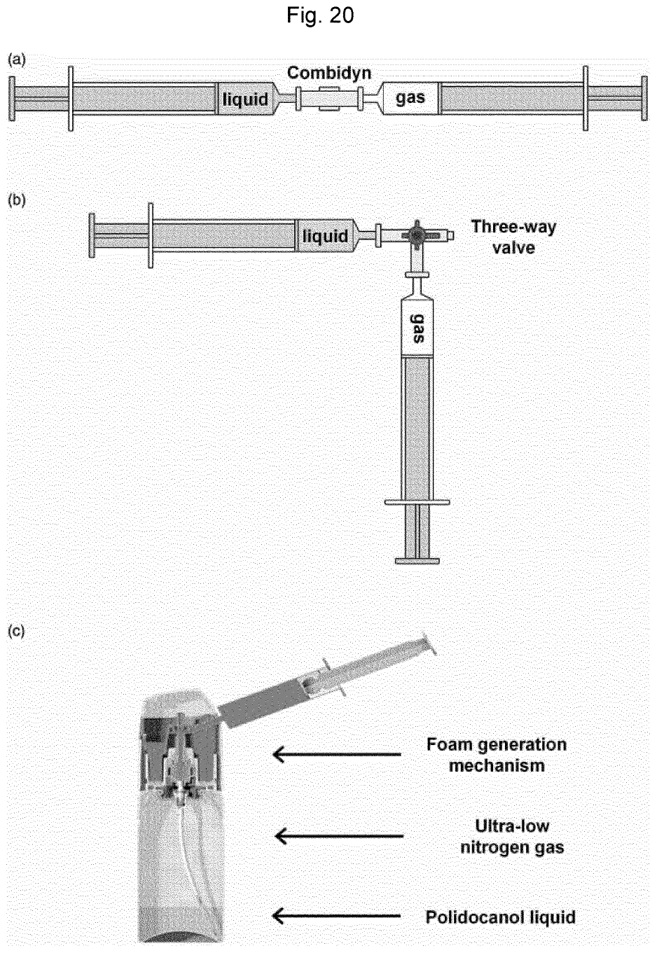

FIG. 20 Schematic drawing of three different devices for generating microfoam: (a) The double syringe system (DSS). DSS involves passing the sclerosant liquid and gas between two syringes joined by a simple straight connector. (b) The Tessari method is similar to DSS, with the difference that the straight connector is replaced with a three-way valve. (c) Polidocanol endovenous microfoam (PEM) is a product generated by a proprietary device called Varithena® (polidocanol injectable foam 1%; Provensis Ltd., a BTG International group company) that produces pharmaceutical-grade low-nitrogen O₂:CO₂ (65:35) foam.

FIG. 1 shows a cross section of the cartridge 100 with the housing 101. As foaming agent reservoir, a syringe 115 is provided within the cartridge 100, connected to the foaming agent reservoir port 125 and delivering the foaming agent through the fluid conduit 126 to the foaming chamber of the reaction vessel 160.

An external gas delivery system 114 is connected with a gas feed port 124 to the gas supply port 134 of the cartridge and delivers the gas through the fluid conduit 121 to the foaming chamber of the reaction vessel 160. An external diluent delivery system 134 is connected with a diluent feed port 123 to the diluent supply port 133 of the cartridge and delivers the diluent through the fluid conduit 120 to the foaming chamber of the reaction vessel 160 which contains a rotatable foaming element 130 situated on the bottom of the foaming chamber.

By thorough mixing of the gas and the foaming agent diluted with the diluent, a microfoam 10 is created which is transported through the fluid conduit 116 to the foam extraction port 170, where it can be retrieved by the user.

The waste material 166 of the foaming process can be removed by transporting it through the fluid conduit 162 to a waste chamber 161.

FIG. 2 shows a cross section of an embodiment for the reaction vessel 160 of the invention as contained within the cartridge 100 of the invention with the housing 101 together with a cross section of the waste chamber 161, which is shown without upper lid.

The reaction vessel 160 defines a cylindrical foaming chamber with a dome-shaped upper part 171 for a more homogenous gas distribution, whereby the gas is supplied through the first inlet channel 172.

A rotating foaming element 130 is provided at the bottom of the foaming chamber and equipped with magnets 132 in order to be magnetically driven by an externally located magnetic stirrer (not shown).

The rotatable foaming element 130 is formed as a disc with a central buckle. The rotatable foaming member 130 contains radially arranged teeth-like protrusions as mixing features 131 which are upwardly bended to provide a defined gap between the teeth and the surrounding reaction vessel.

The foaming chamber contains a deflection element for forced deflection generating a downward fluid motion during the foaming process in direction to the rotating foaming element at the bottom part. In this specific embodiment, the deflection element is formed as a deflection body 141. Said deflection body 141 is situated above the foaming element 130 which is formed as a circular ring segment of trapezoidal basic structure with a beveled side 142 towards the bottom of the foaming chamber and a bottom horizontal side situated above the rotatable foaming element to provide a gap between the deflector body and the upper margin of the rotatable foaming element, whereby circular ring segment is attached to the cylindrical wall of the foaming chamber, and has a radial ring thickness that covers the mixing features 131 of the rotatable disc 130.

The foaming chamber 160 is connected via the second outlet channel 159 and the valve 163 to the waste inlet 164 of the waste chamber 161.

FIG. 3 shows a cross section of an embodiment for the reaction vessel 160 of the invention as contained within the cartridge 100 of the invention with the housing 101. The reaction vessel 160 defines a cylindrical foaming chamber with a dome-shaped upper part 171 for a more homogenous gas distribution, whereby the gas is supplied through the first inlet channel 172 and the aqueous diluent is supplied through the second inlet channel 173.

A rotatable foaming element 130 is provided at the bottom of the foaming chamber with a central axis as positioning member 140 and equipped with magnets 132 in order to be magnetically driven by an externally located magnetic stirrer (not shown).

The rotatable foaming element 130 is formed as a disc with mixing features 131, that are radially arranged teeth-like protrusions which are upwardly bended to provide a defined gap between the teeth and the surrounding reaction vessel.

The foaming chamber contains a deflection element for forced deflection generating a downward fluid motion during the foaming process in direction to the rotating foaming element at the bottom part. In this specific embodiment, the deflection element is formed as a deflection body 141.

Said deflection body 141 is situated above the foaming element 130 which is formed as a circular ring segment of trapezoidal basic structure with a beveled side 142 towards the bottom of the foaming chamber and a bottom horizontal side situated above the rotatable foaming element to provide a gap between the deflector body and the upper margin of the rotatable foaming element, whereby circular ring segment is attached to the cylindrical wall of the foaming chamber, and has a radial ring thickness that covers the mixing features 131 of the rotatable disc 130.

The depicted orientation of the beveled side works with a foaming element that rotates clockwise as seen from above.

The foaming chamber 160 further contains the first outlet channel 174 for removing the microfoam from the foaming chamber 160. Said first outlet channel 174 is located at the bottom of the foaming chamber beneath the deflection body 141.

FIG. 4 shows two possible embodiments of the inventive foaming element 130 and the corresponding restriction arrangement 150.

In FIG. 4A, the restriction arrangement 150 comprises a protrusion 151 to restrict the translatory movement of the foaming element 130. The foaming element 130 comprises extensions 131 towards its outer ends.

In FIG. 4 B, the restriction arrangement 150 further comprises a stop plate 152 to additionally restrict the translatory movement of the foaming element 130.

FIG. 5 shows the cartridge 100 which is viewed from slanted from above after removal of the top lid to give an insight into the housing 101.

The fluid conduits with associated sterile filters and Luer connectors are also removed in order to show the basic elements of the cartridge.

The cartridge housing has a central opening 103 for the syringe plunger 117 (see FIG. 6) of the syringe 115 (see FIG. 6), which functions as foaming agent reservoir.

The housing further contains two fastening members 102, which enable the fastening of the cartridge 100 to the cartridge holder 99 (see FIG. 9).

At the bottom of the housing, there are three female connectors 105 that engage with respective guiding elements 106 (see FIG. 9) of the cartridge holder 99 in order to lock the cartridge in a precisely defined position that secures the safe transport of the gas and the diluent from the automated device 1 (see FIG. 10) to the cartridge 100.

The housing contains the diluent supply port 133 for the supply of the aqueous diluent from the automated device to the cartridge and which is connected to the diluent feed port 123 (see FIG. 9) of the cartridge holder 99.

The housing further contains the gas supply port 134 for the supply of the gas or gas mixture from the automated device 1 to the cartridge 100 and which is connected to the gas feed port 124 (see FIG. 9) of the cartridge holder 99.

The cartridge further contains the reaction vessel 160 with its first inlet channel for gas supply 172 at the top, the second inlet channel for supply of the aqueous diluent 173 and the third inlet channel for supply of the foaming agent located at the bottom of the foaming chamber and controlled by the underlying valve 136.

Besides the reaction chamber 160, a large waste container 161 is provided which is hereby pictured without the upper part of the walls and without the top lid in order to show the internal structures such as the inlet of the waste container 161 which is formed as an opening in an vertical hollow tube, whereby the flow of the waste material is controlled by an underlying valve 163.

FIG. 6 shows the cartridge 100 which is viewed from slanted from above after removal of the top lid to give an insight into the housing 101.

The elements as described in FIG. 5 are also given here.

In addition, FIG. 6 shows the fluid network of the cartridge with associated elements as follows:

The aqueous diluent entering the cartridge through the diluent supply port 133 is guided through the sterile filter 129 which is connected by a Luer connector 122 to the fluid conduit 120 for the diluent transport. Said fluid conduit transports the diluent to the second inlet channel 173 which is connected to said conduit by another Luer connector 122.

The gas or gas mixture entering the cartridge via the gas supply port 134 flows via the fluid conduit 121, via a third Luer connector 122 and via a second sterile filter 129 to the first inlet channel of the foaming chamber of the reaction vessel 160.

The microfoam as generated in the foaming chamber of the reaction vessel 160 is removed from the cartridge via the foam extraction port 170.

On the top of the waste container 161 there is a gas release 165 equipped with a third sterile filter 129 to filter the gas that leaves the waste container and enters the interior of the cartridge.

The cartridge further contains the syringe 115 with its plunger 117. The syringe 115 forms a foaming agent reservoir and is connected to the foaming agent reservoir port 125 connected via the T-piece 128 to the third inlet channel of the foaming chamber 175 and the foaming agent supply port 127.

The plunger 117 penetrates the opening 103 of the cartridge housing 101 and protrudes from the cartridge in order to penetrate the respective opening 104 (not shown) in the cartridge holder 99 (not shown) in order to by pushed by a respective die of the automated device 1 (not shown).

FIG. 7 shows the cartridge 100 viewed in front perspective view after removal of the complete housing 101 of the cartridge to show the interior elements of the cartridge.

The elements as described in FIG. 6 are also given here and the respective description also applies here. The present perspective also shows the diluent supply port 133 as connected to the sterile filter 129 and furthermore the T-piece 128 for the distribution of the foaming agent.

FIG. 8 shows an embodiment for the cartridge in back view and side view after removal of the syringe 115.

The cartridge housing has a central opening 103 for the syringe plunger 117 (not shown) of the syringe 115 (not shown), which functions as foaming agent reservoir.

The housing further contains two fastening members 102, which enable the fastening of the cartridge 100 to the cartridge holder 99 (not shown).

In addition, the cartridge contains the engagement noses 107 for engaging with respective engagement sockets 108 (not shown) provided in the cartridge holder 99 (not shown).

Located on the back, there is the gas supply port 134.

FIG. 9 shows an embodiment for the cartridge holder 99 of the automated device 1, which is viewed in front view (A) and back view (B) both viewed at an angle from above without the cartridge being inserted.

The bottom plate of the cartridge holder contains three male connectors 106 securing the precise position of the cartridge by engaging with the respective three female connectors 105 provided in the bottom of the cartridge 100 (both not shown here).

The bottom plate of the cartridge holder contains the diluent feed port 123 for supplying the gas or gas mixture from the automated device 1 to the cartridge 100.

Within the bottom plate, there is provided a compression die 137 for controlling the valve 136 which is located on the outer bottom surface of the cartridge and controls the flow of the foaming agent.

Within the bottom plate, there is provided a compression die 167 for controlling the valve 163 which is located on the outer bottom surface of the cartridge and controls the flow of the waste material from the foaming chamber to the waste container. The cartridge holder has the opening 104 for insertion of the syringe plunger 117 (not shown here).

In the lower part of the walls of the holder 99, there are three engagement sockets 108 for engaging with the respective engagement noses 107 of the cartridge 100 (both not shown here).

FIG. 10 shows the flow path for the aqueous diluent within the automated device 1 of the invention including the cartridge 100.

FIG. 10A shows the main part of the automated device 1 in front view, whereby the front cover is removed to show the diluent delivery system 113.

A saline infusion bag 180 is installed within the automated device. An silicone tube is connected to said saline bag, from which the diluent is transported to the rotating peristaltic pump (arrow a) to the cartridge holder (arrow b) and further on to the cartridge 100 inserted in the automated device 1.

As shown in (B) the diluent enters (arrow c) the cartridge via the diluent supply port 133.

As shown in (C), the diluent is transported in the interior of the cartridge towards the back part of the cartridge (arrows d and e), where a capacitive sensor in the automated device (not shown here) detects the presence of the liquid within the fluid conduit. The capacitive sensor is arranged to control the fluid flow for the potential presence of air bubbles. The area of measurement is depicted by the dotted ellipse 176.

The diluent is finally transported to the second inlet channel 173 (arrow f) where it enters the foaming chamber of the reaction vessel.

FIG. 11 shows the flow path for the gas within the cartridge 100.

FIG. 11A shows the cartridge in back view, whereby the gas or gas mixture coming from the automated device 1 (not shown here) enters the cartridge via the gas supply port 134 (arrow h). The gas is then transported via the fluid conduit to transported to the first inlet channel 172 (arrow j) where it enters the foaming chamber of the reaction vessel.

After finalizing the foaming process including foam removal, the residual waste in the foaming chamber can be transported by a further gas flow to the waste container, whereby a mixture of gas and the waste enters the waste container by the waste inlet 164 (arrow k).

FIG. 12 shows a flow diagram depicting the process step for the microfoam generation using an automated device 1 and cartridge 100 according to one embodiment of the present invention.

Optional steps are depicted by dotted rectangles.

In step 1, the saline tube is primed and saline and gas are pumped into the foaming chamber of the reaction vessel.

In an optional step 2, the foaming chamber can be cleaned by rinsing the foaming chamber with a saline and/or flushing with gas.

Thereafter, in step 3, defined amounts of saline and foaming agent together with a gas or gas mixture of defined gas ratio are supplied in the foaming chamber.

In the subsequent step 4 defining the initial foaming process, the foaming element is set into rotation reaching (preferentially stepwise) the maximum rotational speed.

In the main foaming process according to step 5, the maximum rational speed of the foaming element is maintained for a defined period of time to generate a microfoam with the target parameters.

In step 6, the rotation speed is reduced to allow the extraction of the foam as supported by establishing a slight over pressure in the foaming chamber, so that the user can remove in step 7 the microfoam from the foaming chamber.

Optionally, the user can remove further portions of microfoam according to step 8.

Notably, the automated device allows a repeated execution of the microfoam generation process, so that after steps 7 or 8 a further foam generation process can be initiated by starting with step 1 again.

FIG. 13 depicts cross sections of a cartridge according to one embodiment of the invention showing cleaning steps for the foaming chamber.

As shown in (A) a rinsing of the foaming chamber with saline entering the second inlet channel 173 and flushing with gas/gas mixture entering the first inlet channel 17, the foaming chamber is cleaned. Hereby, the gas or gas mixture as intended for the foaming process should be also used for the cleaning step in order to equilibrate the foaming chamber with the final gas or gas mixture.

As shown in (B), the gas and the saline together with potential residues form a prior foaming process are removed from the foaming chamber by further introducing the gas or gas mixture into the foaming chamber while opening (m) the valve transport valve 163, so that the residues are transported from the foaming chamber (arrow l) through the second outlet channel 159 (arrow m) to enter the waste container 161 via the waste inlet (arrow n).

FIG. 14 depicts cross sections of a cartridge according to one embodiment of the invention showing steps preparing the foaming chamber for the foaming process.

As shown in (A), the defined gas or gas mixture is introduced via the first inlet channel 172 into the foaming chamber, while the valve 163a is opened to allow the gas or gas mixture, respectively to leave the foaming chamber via the second outlet channel to enter the waste container. As a result, the foaming chamber is equilibrated with the defined gas or gas mixture which should be used for the preparation of the microfoam.

In the next step as shown in B, the valve 163b is closed and a defined amount of saline is introduced into the foaming chamber via the second inlet channel 173, and also a defined amount of foaming agent is introduced into the foaming chamber via the third inlet channel 175.

A temporarily opening of a venting valve, which is part of the gas distribution system of the automated device 1 (not shown here) prior the introduction of the diluent and the foaming agent prevents an overpressure within the foaming chamber during the supply of the diluent and the foaming agent.

As a result, the foaming chamber is provided with all necessary components of the foaming process, namely the foaming agent, the aqueous diluent and also the gas or gas mixture.

FIG. 15 shows a schematic drawing of a cross section of a cartridge according to one embodiment of the invention showing the step of microfoam removal from the foaming chamber.

By opening the valve 136 and introducing the defined gas or gas mixture via the first inlet channel 172 into the foaming chamber in order to generate a slight “foam extraction over-pressure” within the foaming chamber, the microfoam can be removed through the first outlet channel, preferably by introducing a syringe through the valve of the foam extraction port and aspirating the microfoam.

A slight “foam extraction over-pressure” as understood herein, refers to an over-pressure of 20 mbar or less and preferably of 10 mbar or less.

FIG. 16 displays a conceptual view of an embodiment of a user interface 200.

It is to be noted that this embodiment solely represents a graphical user interface to which an inventive user interface may not be limited as it may further comprise elements like a screen, particularly a touch screen, mechanical buttons or an interface like a keyboard or mouse.

The user interface 200 in FIG. 16 comprises several graphical control elements 202 that may provide functions like cancelling a computer program and/or continuing a computer program and/or moving one step backwards in a computer program.

The user interface 200 comprises several graphical elements 201 that may allow a selection of at least one parameter regarding the foaming process like a concentration of an agent solution or at least one parameter regarding the user interface 200 like a language.

The user interface 200 comprises two graphical sections 203. In one of the graphical sections 203 several informational images 206 are provided that may show parameters regarding the foaming process that were selected by the user or that are describing a current state of the foaming process. In the other graphical section 203 an instructional image 205 is provided. The instructional image 205 may show a user how to mount the cartridge or how and/or where to provide an agent solution or how to clean the cartridge or how and/or where to extract the inventive microfoam 10 after the foaming process.

The graphical control elements 202 as well as the graphical elements 201 may be controlled or actuated by a touch screen or by clicking with a mouse or by using a keyboard or mechanical buttons.

FIG. 17 shows another conceptual view of an embodiment of an inventive user interface 200. As mentioned in the preceding paragraph, the user interface may not be limited to a graphical user interface like the one in FIG. 16 and FIG. 17.

The user interface 200 in FIG. 17 comprises a graphical section 203 containing several informational images 206.

The user interface 200 further comprises a graphical element 201 as well as two graphical control elements 202.

The aforementioned graphical element 201 as well as the two graphical control elements 202 may provide the same functionalities as was described in regard to FIG. 16.

The user interface 200 in FIG. 17 also comprises several foaming profiles 204. This may indicate that a user may select a specific foaming profile 204 out of the ones that are provided which may be in accordance with specific applicational requirements, particularly medical requirements. The selection of the foaming profile may be provided by swiping and/or tapping on a touch screen or by clicking with a mouse or by using a keyboard or mechanical buttons.

FIG. 18 shows the bubble distribution for a microfoam according to the invention. The measurements were recorded 45 sec (A) or 115 sec (B) after foam preparation for the same microfoam.

The microfoam was prepared as follows: The microfoam was generated with a cartridge as shown in FIGS. 13 to 15 at room temperature. After flushing the foaming chamber with a gas mixture of 80% oxygen and 20% carbon dioxide, 2 ml of an aqueous 2% Polidocanol solution as and 18 ml of an isotonic 0.8% sodium chloride solution were injected into the cartridge.

Afterwards, the rotor of the cartridge is driven by an external magnetic stirrer according to the following Reaction settings:
1. Prefoaming: Ramp of 10 seconds from 0 to 2.000 rpm, and holding at 2.000 rpm for 20 seconds;
2. Foaming: Ramp of 10 seconds from 2.000 rpm to 4.000 rpm and holding at 4.000 rpm for 40 seconds;
3. Extraction phase: Maintaining the rpm at 4.000 rpm for up to several minutes.

The foaming chamber of the cartridge comprises a pressure compensation device for compensating for pressure fluctuations in the pressure within the foaming chamber. The process generates approximately 20 ml of microfoam having a moisture content of 15.9±2.01% (w/w). Thereafter, the freshly generated microfoam is extracted from the foam supply port of the cartridge by use of a syringe.

FIG. 19(A) shows the bubble distribution for PEM, Tessari and DSS (double syringe system) samples as shown in FIG. 3 of Roberts et al, 2020 (Biorheology; 57 (2-4): 77-85). For preparation of PCFs, 1% aqueous buffered polidocanol solution was used for all three microfoams. The mean bubble size given in μm is an average over the foam, i.e. the sum of the bubble radii divided by the number of bubbles. Instead of this unweighted mean, it is known that hat the Sauter mean radius, is the most appropriate average bubble size for predicting the rheological properties of foams. The Sauter mean radius approximates the average bubble size based on the ratio of volume to surface area. In particular, the Sauter mean radius is sensitive to the presence of any large bubbles, recognizing that a long tail in the bubble size distribution has a significant effect on foam response.

It is for this reason that a narrow bubble size distribution is more appropriate for sclerotherapy.

The three microfoams were generated as follows:

The DSS foam was produced by passing 1 mL polidocanol from a 5 ml syringe (Discardit™ II, Becton Dickinson, Erembodegem, Belgium), ten times into and out of a 10 ml syringe containing 7 mL of gas. Syringes were interfaced via a straight connector (equivalent, Female-to-Female Luer Lock Connector, QOSINA Edgewood, NY, USA).

For the Tessari method, the straight connector is replaced with a 3-way valve (BD Connecta™ 3-Way Stopcocks, Becton Dickinson, Erembodegem, Belgium). A further modification involves setting the valve tap at a 30° angle to increase shear when passing the foam between the syringes, which was adopted in the present study. As with the DSS method, 5 and 10 mL syringes were used to prepare the foam. PEM is a combination drug device product in development by Provensis Ltd. (a BTG International Group Company, London, UK) consisting of a proprietary 65:35 O₂:CO₂ gas mixture with ultralow nitrogen content (<0.8%) and 1% polidocanol solution (no additional stabilizers are added), contained within a pressurized canister and combined on discharge from the canister as a uniform microfoam. Sterile canisters of the product were used as per the instructions for use (IFU), to generate 5 mL of microfoam for experimentation.

The microfoam was drawn from the canister via a Microfoam Transfer Unit (MTU) into a 10 mL Norm-Ject syringe (Henke-Sass Wolf, Tuttlingen, Germany).

As shown in the table of FIG. 19 (B), the device according to the present invention produces a superior microfoam exhibiting a dramatic reduction in mean bubble size and the Sauter mean radius.

This is even more remarkable given the fact that the microfoam of the invention was produced with a reduced concentration of polidocanol (0.2% versus 1%), since a reduction in the concentration of the foaming agent makes it more difficult to create a microfoam of sufficient stability.

FIG. 20 shows three devices for producing microfoam.

In the DSS method, syringes are connected by a Combidyn® adapter (a), while in the Tessari method, they are connected by a three-way valve (b). In both techniques, the foam was produced by passing the polidocanol solution (liquid phase) from one syringe, 10 times into and out of the other syringe initially containing the gas or gas mixture (gaseous phase). Foam was produced at room temperature (20° C.-22° C.). The proprietary canister system for generating PEM (Varithena®) is shown in (c).

The foregoing explanation of the embodiments describes the present invention exclusively in the context of examples. Of course, individual features of the embodiments can be freely combined with each other, provided that this is technically reasonable, without leaving the scope of the present invention.

It shall be underlined that—in addition to the claims of the present patent application—the following feature combinations are also advantageous. These feature combinations are to be understood as potentially replace the patent claims, or to be added to the patent claims:

1. Reaction vessel for producing a microfoam that contains a reaction unit body defining a foaming chamber and containing:
a. a rotatable foaming element arranged in the foaming chamber for performing a foaming process to produce the microfoam from a liquid foaming composition,
b. a positioning member on the bottom of the foaming chamber that holds the foaming element to be rotatable around a predefined axis during the foaming process,
c. a first outlet channel provided adjacent to or at the bottom part of the foaming chamber that is in fluid communication with the said foaming chamber for removing the microfoam from the foaming chamber.

2. Reaction vessel according to feature combination 1, characterized in that the reaction unit body defining the foaming chamber further has one or more of the following characteristics, and preferably all of the following characteristics:
a. a first inlet channel that is in fluid communication with the said foaming chamber for supplying a gas or gas mixture to the foaming chamber, whereby the first inlet channel is preferably provided at the top part of the foaming chamber,
b1. a second inlet channel that is in fluid communication with the said foaming chamber for supplying a mixture comprising a foaming agent and an aqueous diluent to the foaming chamber, whereby the second inlet channel is preferably provided adjacent to or at the bottom part of the foaming chamber, or
b2. a second inlet channel that is in fluid communication with the said foaming chamber for supplying an aqueous diluent to the foaming chamber, whereby the second inlet channel is preferably provided at the top part of the foaming chamber, provided together with a third inlet channel that is in fluid communication with the said foaming chamber for supplying a foaming agent to the foaming chamber, whereby the third inlet channel is preferably provided adjacent to or at the bottom part of the foaming chamber.

3. Reaction vessel according to feature combination 1 or 2, characterized in that the reaction unit body defining the foaming chamber further has a second outlet channel that is in fluid communication with the said foaming chamber for removing waste material from the foaming chamber whereby the second outlet channel is preferably provided adjacent to or at the bottom part of the foaming chamber.

4. Reaction vessel according to any of the preceding feature combinations, characterized in that the reaction unit body defining the foaming chamber further has a deflection element for forced deflection generating a downward fluid motion during the foaming process in direction to the rotating foaming element at the bottom part.

5. Reaction vessel according to any of the preceding feature combinations, characterized in that the reaction unit body is a sealed reaction unit body and/or a sterile or sterilizable reaction unit body.

6. Reaction vessel according to any of the preceding feature combinations, characterized in that the rotatable foaming element is configured as a disc with mixing features suitably shaped for inducing a turbulent stream and located around an outer perimeter of said disc, whereas the mixing features comprise one or more of the following: blades, rings, teeth, fins, rips, wings, arms, fingers, perforated plates, grid structures, or a ring-shaped helical spring.

7. Reaction vessel according to any of the preceding feature combinations, characterized in that the rotatable foaming element, being preferably a disc, is configured to be magnetically driven by a magnetic stirrer located outside the foaming chamber and preferably beneath the bottom part of the foaming chamber.

8. Reaction vessel according to any of the preceding feature combinations, characterized in that the rotatable foaming element has a centrally located bore, which is preferably open at one end for receiving the positioning member.

9. Reaction vessel according to any of the preceding feature combinations, characterized in that the positioning member is a centrally or eccentrically located shaft on the bottom of the foaming chamber and preferably has a cylindrical side wall with a rounded upper corner.

10. Reaction vessel according to any of the preceding feature combinations, characterized in that the reaction vessel further comprises a restriction member for restricting an upward movement of the rotatable foaming element, whereby said restriction member is preferably a rod-shaped element extending from the top part of the foaming chamber (Advantage: will not fall off the positioning member during transport and handling).

11. Reaction vessel according to any of the preceding feature combinations, characterized in that the deflection element is a deflector plate or deflector body located in the foaming chamber and/or a propellor provided on the rotating member.

12. Reaction vessel according to feature combination 11, characterized in that the deflector body is a circular ring segment of trapezoidal basic structure with a beveled side towards the bottom of the foaming chamber and a bottom horizontal side situated above the rotatable foaming element to provide a gap between the deflector body and the upper margin of the rotatable foaming element, whereby circular ring segment is preferably attached to the cylindrical wall of the foaming chamber, having more preferably a radial ring thickness that substantially covers the mixing features of the rotatable disc.

13. Reaction vessel according to feature combination 12, characterized in that the first outlet channel is situated under the bottom horizontal side of the deflector body.

14. Reaction vessel according to any of feature combinations 2 to 13, characterized in that the reaction unit body contains a gas distributor system, preferably selected from:
a. a deviation plate or body located in the foaming chamber beneath the first inlet channel to induce a lateral deflection of the gas stream entering the foaming chamber, or
b. a semispherical-, conical-, frustoconical- or dome-shaped top part of the foaming chamber with the first inlet channel at the top enabling a gas flow which is uniformly distributed over the horizontal cross-section of the foaming chamber.

15. Reaction vessel according to any of the above feature combinations, characterized in that the surface of an upper section of the foaming chamber is coated with a hydrophobic or superhydrophobic agent or is a morphologically structured (super) hydrophobic surface such as a superamphiphobic, a slippery liquid-infused porous surface (SLIPS), or a liquid-impregnated surface (LIS).

16. Cartridge containing
a. a reaction vessel according to any of feature combinations 1 to 15,
b. a foam extraction port containing a valve and arranged accessible from an outside of the cartridge for extraction of the produced microfoam, and
c. a fluid conduit coupling the first outlet channel of the foaming chamber with the said foam extraction port.

17. Cartridge according to feature combination 16 further containing
a. a waste container defining a waste chamber containing an inlet for receiving the waste material from the foaming chamber,
b. a fluid conduit coupling the second outlet channel of the foaming chamber through a valve with the said inlet of the waste container, and
c. the waste container optionally contains an outlet for pressure release, whereas the cartridge according to feature combination 16 contains a reaction vessel according to any of feature combinations 3 to 15.

18. Cartridge according to feature combination 17, characterized in that the inlet of the waste container is positioned in the top part of the waste container, preferably as the upper aperture of a hollow shaft positioned preferably in the center of the waste chamber.

19. Cartridge according to feature combination 17 or 18, characterized in that the valve for controlling the flow of the waste material into the waste chamber is located on the outer surface or accessible from an outer surface of the cartridge and can be controlled by exerting an external pressure on said valve.

20. Cartridge according to feature combination 17 to 19, characterized in that the inner surface of the waste chamber is coated with a defoaming agent or has a morphologically structured defoaming inner surface such as an anti-foaming surface such as a superamphiphobic, a slippery liquid-infused porous surface (SLIPS), or a liquid-impregnated surface (LIS).

21. Cartridge according to feature combination 17 to 20, characterized in that the cartridge further contains:
a. a gas supply port containing a sealing member and arranged accessible from an outside or located on the outer surface of the cartridge for supply of a gas or gas mixture.
b. a fluid conduit coupling the said gas supply port and the first inlet channel of the foaming chamber.

22. Cartridge according to feature combination 17 to 21, characterized in that the cartridge further contains:
a. a diluent supply port containing a sealing member and arranged accessible from an outside of the cartridge for supply of an aqueous diluent,
b. a fluid conduit coupling the said diluent port and the second inlet channel of the foaming chamber,
c. a foaming agent reservoir, being preferably a syringe, enabling a fluid tight seal with a foaming agent reservoir port.

23. Cartridge according to feature combination 22, characterized in that the cartridge further contains:
a. a foaming agent supply port containing a valve and arranged accessible from an outside of the cartridge for supply of a foaming agent,
b. a fluid conduit coupling the said foaming agent supply port with the foaming agent reservoir port and with the third inlet channel of the foaming chamber, wherein said fluid conduit contains a valve which is positionable to allow a fluid communication of the foaming agent reservoir port with the foaming agent supply port or alternatively of the foaming agent reservoir port with the third inlet channel, and wherein said valve is preferably switchable by exerting an external pressure on the cartridge.

24. Cartridge according to feature combination 16 to 23, characterized in that the cartridge further contains means for uniquely identifying the cartridge and/or its properties, preferably selected from an RFID tag, a barcode, a data matrix code, a QR code or a similar code.

25. An automated device for generating microfoam containing:
a. a cartridge holder adapted to hold a replaceable cartridge to any of feature combinations 16 to 24,
b. a magnetic stirrer situated beneath the cartridge for generating a rotating magnetic field for controlling the rotation of the foaming element around the positioning member.

26. The automated device according to feature combination 25, characterized in that the cartridge holder contains a gas feed port that is configured to fluidly engage with the gas supply port of the cartridge in the holder, thereby providing a gas-tight seal of both ports, wherein the gas feed port or the system preferably contains a safety valve allowing a gas supply only for a cartridge being properly locked in the cartridge holder.

27. The automated device according to feature combination 25 or 26, characterized in that the cartridge holder contains a diluent feed port that is configured to fluidly engage with the diluent supply port of the cartridge in the holder, thereby providing a fluid-tight seal of both ports, wherein the diluent feed port or the system preferably contains a safety valve allowing a diluent supply only for a cartridge being properly locked in the cartridge holder.

28. The automated device according to feature combination 25 to 27, characterized in that the automated device contains a sensor that is configured to detect within the cartridge the presence or flow of a liquid in the fluid conduit coupling the diluent port and the second inlet channel of the foaming chamber, whereby said sensor is preferably a capacitive sensor or an ultrasound sensor.

29. The automated device according to feature combination 25 to 28, characterized in that the cartridge holder contains one or more guiding elements for proper insertion of the cartridge within the cartridge holder, wherein the guiding device preferably contains at least two retractable members arranged at the bottom of the cartridge holder and mate with respective orifices of the cartridge housing.

30. The automated device according to feature combination 25 to 29, characterized in that the cartridge holder contains a fastening mechanism configured to fasten the cartridge to the cartridge holder, whereby the fastening mechanism preferably contains one or more members that are arranged in the lateral wall of the cartridge holder and can be extended to engage with respective features of the cartridge housing.

31. The automated device according to feature combination 25 to 30, characterized in that the automated device contains a gas supply system containing:
a. at least one gas port for connection to at least one gas container, being preferably a gas bottle,
b. at least one gas conduit connecting the at least one gas port to the gas supply port via a valve for regulating the gas flow,
c. optionally a pressure compensation device for compensating for pressure transients in the pressure within the foaming chamber.

32. The automated device according to feature combination 25 to 31, characterized in that the automated device contains a gas supply system containing
a. two gas ports for connection to two gas bottles containing different gases, preferably installed in the automated device and being more preferably an O₂ gas bottle and a CO₂ gas bottle,
b. a gas channel network between the two gas ports and the gas feed port,

c. a gas mixing device for mixing the two different gases in a selectable ratio, and
d. optionally a pressure compensation device for compensating for pressure transients in the pressure within the foaming chamber.

33. The automated device according to feature combination 25 to 32, characterized in that the automated device contains a diluent supply system containing
a. a diluent port for connection to a diluent container, preferably installed in the automated device, and
b. a liquid conduit connecting the diluent port to the diluent feed port via a pump capable of transporting the diluent from the diluent container to the diluent feed port.

34. The automated device according to feature combination 25 to 33, characterized in that the automated device contains a syringe-associated delivery system containing a drive unit moving an agitator which can perform at least one of the following actions:
a. detecting the correct connection of the inserted syringe to the foaming agent reservoir port of the said holding member,
b. detecting the complete filling of the syringe via the foaming agent reservoir port by detecting the final position of the plunger,
c. delivering a determined amount of foaming agent to the foaming chamber by pushing the plunger of the foaming agent-containing syringe to a controlled extent,
d. optionally detecting malfunctions of the syringe by determining the amount of pressure to be executed on the plunger of the syringe.

35. The automated device according to any of feature combinations 25 to 34, characterized in that the system further comprises:
a. a control computing unit, in which the control program is located connected with a user interface for controlling the foaming process, particularly by configuring at least one parameter of the foaming process and/or by initiating the foaming process, the at least one parameter being directly or indirectly selected from the following:
i. a gas concentration,
ii. a mixing ratio of the gas mixture,
iii. a concentration of the foaming agent in the mixture of foaming agent and diluent,
iv. the total amount of the mixture of foaming agent and diluent, selection of the liquid composition,
v. the rotation speed and time of the rotatable foaming element,
vi. the removal of waste material from the foaming chamber,
vii. the cleansing of the foaming chamber as preparation for a further foaming process,
viii. the removal of the cartridge from the cartridge holder,
   wherein the automated device is configured to perform the foaming process based on the configured parameter automatically, and particularly the mixing of the gases according to the configured mixing ratio of the gas mixture automatically.

36. A method for automatically producing a microfoam comprising the following steps:
a. providing the reaction vessel according to any of feature combinations 3 to 15,
b. optionally flushing the foaming chamber with a selected gas or gas mixture or rinsing with the aqueous diluent, being preferably saline,
c. introducing the selected gas or gas mixture into the foaming chamber via the first inlet channel,
d. introducing a mixture of the foaming agent and the aqueous diluent into the foaming chamber via the second inlet channel, or alternatively, introducing the aqueous diluent into the foaming chamber via the second inlet channel and the foaming agent into the foaming chamber via the third inlet channel,
e. rotating the magnetic stirrer to rotate the foaming element until a suitable microfoam has been obtained,
f. removal of the obtained therapeutical foam from the foaming chamber via the first outlet channel.

37. A method for automatically producing a microfoam comprising the following steps:
a. inserting a cartridge according to any of feature combinations 16 to 24 into the cartridge holder of the automated device according to any of feature combination 25 to 35,
b. selecting a foaming agent concentration using a user interface,
c. selecting a ratio for the mixture of two gases using a user interface,
d. Filling the foaming agent reservoir by injecting a foaming agent through the sealing member of the foaming agent supply port,
e. Starting the foam generation process using a user interface so that the system automatically rotates the magnetic stirrer to rotate the foaming element in the foaming chamber of the cartridge until a suitable microfoam has been obtained,
f. Once the foam has been generated removal of the microfoam by extraction from the foam supply port of the cartridge,
g. Optionally initiating a cleansing process of the foaming chamber by using a user interface,
h. Optionally repeating the process according steps a. to g.

38. A microfoam obtainable by a method according to feature combination 36 or 37.

39. The microfoam according to feature combination 38, characterized in that the microfoam has a nitrogen content of less than 2%, preferably of less than 1% and more preferably of less than 0.8%.

40. The microfoam according to feature combination 38 or 39, characterized in that the microfoam has a number of geometrical impurities being less than 30% of the bubble count, preferably less than impurities being less than 10% of the bubble count and more preferably less than 1% of the bubble count.

41. The microfoam according to feature combination 38 to 40, characterized in that the microfoam has a mean bubble size of less than 100 μm, preferably less than 80 μm, and more preferably less than 50 μm, measured 45 seconds after foam preparation.

42. The microfoam according to feature combination 38 to 41, characterized in that the microfoam has a Sauter mean radius of less of less than 200 μm, preferably less than 150 μm, and more preferably less than 100 μm, measured 45 seconds after foam preparation.

43. The microfoam according to feature combination 38 to 42, characterized in that the microfoam has a moisture content of less of less than 20% (w/w), being preferably between 8 and 12% (w/w).

44. A computer program comprising instructions for performing steps of feature combinations 36 or 37, which, when the program is executed by a computer, cause the computer to carry out the following steps:
a. Providing a user interface for controlling a foaming process for producing a microfoam, particularly by configuring at least one parameter of the foaming process and/or by initiating the foaming process, the at least one parameter being selected from the following: a gas concentration, a mixing ratio of the gas mixture, a selection of the liquid composition, a mixing ratio of the agent solution, a concentration of the agent solution in the microfoam, a foaming profile (204),
b. providing at least one graphical element (201) as part of the user interface (200) for selecting or configuring the at least one parameter,
c. providing at least one graphical control element (202) for directing the computer program, in particular a graphical control element (202) for cancelling the computer program and/or a graphical control element (202) for continuing the computer program and/or a graphical control element (202) for moving one step backwards in the computer program,
d. providing at least one graphical section (203) as part of the user interface (200) showing at least one instructional image (205) and/or at least one informational image (206) for guiding or informing a user.

45. Computer program according to feature combination 44, characterized in that the foaming profile (204) comprises at least one of the following parameters:
profile name and description,
names of foaming agent, diluent, gasses,
list of compatible cartridges,
available selection of agent input concentration,
valid range of agent input concentration,
volume of agent to be injected into the cartridge,
available selection of agent concentration in foam,
available selection of gas ratios,
detailed instructions of how to prepare the microfoam,
limit of keeping the microfoam fresh after preparation,
time limit for use of cartridge.

### LIST OF REFERENCE NUMERALS

1 Automated device
10 microfoam
99 Cartridge holder
100 Cartridge
101 Cartridge housing
102 Fastening member on the Cartridge housing
103 Opening in the Cartridge housing for the syringe plunger
104 Opening in the Cartridge holder for the syringe plunger
105 Female connector in the cartridge housing to engage with guiding elements
106 Male connector in the cartridge holder as guiding element
107 Engagement nose for engaging with cartridge holder
108 Engagement socket for engaging with engagement nose 107 of cartridge
113 Diluent delivery system
114 Gas delivery system
115 Syringe as foaming agent reservoir
116 Fluid conduit for microfoam transport
117 Plunger of the syringe
120 Fluid conduit for diluent transport
121 Fluid conduit for gas transport
122 Luer lock connector
123 Diluent feed port
124 Gas feed port
125 Foaming agent reservoir port
126 Fluid conduit for foaming agent
127 Foaming agent supply port
128 T-piece in fluid conduit of foaming agent
129 Sterile filter
130 Rotatable foaming element
131 Mixing features
132 Magnet
133 Diluent supply port
134 Gas supply port
135 Magnetic stirrer device
136 Valve controlling the supply of foaming agent
137 Compression die controlling the valve 136
140 Positioning member
141 Deflection body
142 Beveled side of deflection body
150 Restriction member
151 Protrusion
152 Stop plate
159 Second outlet channel (waste outlet opening) of the foaming chamber
160 Reaction vessel/reaction unit with foaming chamber
161 Waste container defining a waste chamber
162 Fluid conduit for waste transport
163 Valve controlling the waste transport
163a Valve controlling the waste transport is opened
163b Valve controlling the waste transport is closed
164 Inlet of the waste container
165 Gas release of the waste container
166 Waste
167 Compression die controlling the valve 163 for waste transport
170 Foam extraction port
171 Dome-shaped top part of the foaming chamber
172 First inlet channel (gas supply opening) of the foaming chamber for gas supply
173 Second inlet channel (diluent supply opening) of the foaming chamber for
diluent supply
174 First outlet channel (foam extraction opening) of the foaming chamber for
removing microfoam
175 Third inlet channel (foaming agent supply opening) of the foaming chamber for
foaming agent supply
176 Area for controlling the diluent flow in the fluid conduit
180 Saline infusion bag
185 Peristaltic pump
200 User interface
201 Graphical element
202 Graphical control element (touchscreen element)
203 Graphical section
204 Foaming profile
205 Instructional image
206 Informational image

1. Cartridge for housing the producing of a therapeutical microfoam, comprising
a. a housing body,
the housing body comprising a hollow interior; and
b. a reaction unit within the hollow interior,
the reaction unit comprising a reaction unit body,
the reaction unit body comprising a foaming chamber; and
c. a foam extraction port
arranged accessible from an outside of the housing body for extracting produced microfoam; and
d. optionally: a reservoir within the hollow interior comprising a liquid foaming agent, while the cartridge is sealed.

2. Cartridge according to claim 1, wherein the foam extraction port is located at a surface of the housing body, preferably at a top or front surface of the housing body.

3. Cartridge according to any of the preceding claims, wherein the housing body comprises, within the hollow interior, a foam extraction conduit fluidically coupling the reaction unit, preferably the foaming chamber, to the foam extraction port.

4. Cartridge according to the preceding claim, wherein the foam extraction conduit is at least partly constructed as a part of the reaction unit body.

5. Cartridge according to any of preceding claims 2 to 4, wherein the foam extraction conduit comprises a fluidic connection to a foam extraction opening of the foaming chamber, the foam extraction opening being located in a lower half of the foaming chamber, preferably reaching a bottom of the foaming chamber.

6. Cartridge according to any of preceding claim 4 or 5, wherein the foam extraction conduit connects a channel outlet in the reaction unit body with the foam extraction port.

7. Cartridge according to any of preceding claims 3 to 6, wherein the foam extraction conduit comprises a valve.

8. Cartridge according to any of preceding claims 3 to 7, wherein the foam extraction conduit exclusively consists of rigid parts, free of hosing.

9. Cartridge according to any of the preceding claims, wherein the foam extraction port or a foam extraction conduit comprise a foam extraction filter.

10. Cartridge according to any of the preceding claims, wherein the foam extraction port comprises a detachable foam extraction port sealing member and/or a foam extraction conduit valve.

11. Cartridge according to any of the preceding claims, wherein the foam extraction port comprises a foam extraction reservoir.

12. Cartridge according to any of the preceding claims, wherein the foam extraction port comprises a syringe dock.

13. Cartridge according to any of the preceding claims, wherein the cartridge further comprises a gas supply port
which is in case of one of claims 2 to 12 different from the foam extraction port.

14. Cartridge according to preceding claim 13, wherein the gas supply port is located at a surface of the housing body, preferably at a bottom or back surface of the housing body.

15. Cartridge according to any of preceding claim 13 or 14, wherein the housing body comprises, within the hollow interior, a gas supply conduit, fluidically coupling the gas supply port to the reaction unit, preferably to the foaming chamber.

16. Cartridge according to preceding claim 15, wherein the gas supply conduit comprises a gas supply opening of the foaming chamber, the gas supply opening being located in an upper half of the foaming chamber, preferably on a top lid of the foaming chamber.

17. Cartridge according to any of preceding claim 15 or 16, wherein the gas supply conduit comprises a hose, preferably a translucent hose.

18. Cartridge according to any of preceding claims 15 to 17, wherein the gas supply conduit comprises a pressure relieve.

19. Cartridge according to any of preceding claims 13 to 18, wherein the gas supply port or a gas supply conduit comprise a gas supply filter.

20. Cartridge according to any of preceding claims 13 to 19, wherein the gas supply port comprises a detachable gas supply port sealing member.

21. Cartridge according to any of preceding claims 13 to 20, wherein the cartridge comprises exactly one gas supply port.

22. Cartridge according to any of preceding claims 13 to 20, wherein the cartridge comprises two gas supply ports, and wherein within the interior of the housing body, there is a merging junction for gas coming from the two gas supply ports.

23. Cartridge according to any of the preceding claims, wherein the cartridge further comprises a diluent supply port which is in case of claims 2 to 12 apart from the foam extraction port and/or which is in case of claims 13 to 22 apart from the gas supply port.

24. Cartridge according to preceding claim 23, wherein the diluent supply port is located at a surface of the housing body, preferable at a bottom or back surface of the housing body.

25. Cartridge according to preceding claim 23 or 24, wherein the housing body comprises, within the hollow interior, a diluent supply conduit, fluidically coupling the diluent supply port to the reaction unit, preferably to the foaming chamber.

26. Cartridge according to preceding claim 25, wherein the diluent supply conduit comprises a diluent supply opening being located in an upper half of the foaming chamber, preferably on a top lid of the foaming chamber.

27. Cartridge according to any of preceding claims 24 to 26, wherein the saline supply conduit comprises a hose, preferably a translucent hose.

28. Cartridge according to preceding claim 27, wherein the saline supply conduit hose Is longer than a gas supply conduit hose.

29. Cartridge according to any of preceding claims 23 to 28, wherein the saline supply port or a saline supply conduit comprise a saline supply filter.

30. Cartridge according to any of preceding claims 23 to 29, wherein the saline supply port comprises a detachable saline supply port sealing member.

31. Cartridge according to any of the preceding claims, wherein the cartridge further comprises a foaming agent supply port,
which is in case of claims 2 to 12 apart from the foam extraction port and/or which is in case of claims 13 to 22 apart from the gas supply port and/or which is in case of claims 23 to 30 apart from the saline supply port.

32. Cartridge according to preceding claim 31, wherein the foaming agent supply port is located at a surface of the housing body, preferably at a top or front surface of the housing body and/or on the same side of the housing body as the foam extraction port.

33. Cartridge according to any of preceding claim 31 or 32, wherein the housing body comprises, within the hollow interior, a foaming agent supply conduit, fluidically coupling the foaming agent supply port to the reaction unit, preferably to the foaming chamber.

34. Cartridge according to preceding claim 33, wherein the foaming agent supply conduit comprises a foaming agent supply opening being located in an upper half of the foaming chamber, preferably on a top lid of the foaming chamber.

35. Cartridge according to preceding claims 31 to 34, wherein the foaming agent supply port comprises an injection port for receiving a syringe needle.

36. Cartridge according to preceding claims 31 to 35, wherein the housing body comprises, in its hollow interior, a foaming agent reservoir which is fluidically coupled but apart from the foaming agent port and/or which is fluidically coupled but apart from the foaming chamber.

37. Cartridge according to preceding claim 36, wherein the foaming agent reservoir is included in a foaming agent supply conduit for receiving and forwarding the foaming agent in a unidirectional flow path from the foaming agent supply port to the foaming chamber.

38. Cartridge according to preceding claim 36, wherein the foaming agent reservoir is fluidically connected to the foaming agent supply conduit for receiving and forwarding the foaming agent in a bidirectional flow path from the foaming agent supply port to the foaming chamber.

39. Cartridge according to preceding claims 36 to 38, wherein the foaming agent reservoir is a hollow body with an ejector.

40. Cartridge according to preceding claim 39, wherein the ejector is a passive element which is movable by an ejection actor which is located outside the cartridge, preferable the housing body comprising an ejector access opening in its surface, wherein the foaming agent reservoir might be a syringe.

41. Cartridge according to preceding claim 39, wherein the ejector is a passive element which is movable by an ejection actor which is located within the hollow interior of the housing body.

42. Cartridge according to preceding claim 41, wherein the ejection actor is a pneumatic drive.

43. Cartridge according to preceding claim 41, wherein the ejection actor is an electric drive.

44. Cartridge according to any of preceding claims 31 to 43, wherein the cartridge comprises a valve for selectively opening or closing and/or redirecting a fludical path from the foaming agent supply port to the foaming chamber and/or to the foaming agent reservoir.

45. Cartridge according to any of preceding claims 31 to 44, wherein the cartridge comprises a valve for selectively opening or closing and/or redirecting a fludical path from the foaming agent reservoir to the foaming chamber.

46. Cartridge according to any of preceding claims 33 to 45, wherein the foaming agent supply conduit comprises a T-junction.

47. Cartridge according to any of preceding claims 33 to 46, wherein the foaming agent supply conduit comproses a foaming agent filter.

48. Cartridge according to any of preceding claims 31 to 47, wherein the cartridge comprises a detachable foaming agent port sealing member.

49. Cartridge according to any of the preceding claims, wherein the cartridge further comprises, within the reaction unit for producing a microfoam, within the foaming chamber or fluidically connected to the foaming chamber, a foaming element.

50. Cartridge according to preceding claim 49, wherein the foaming element comprises a pressure-filter unit, wherein a filter is provided at a pressure relief so that a sclerosant composition pressed under pressure through the filter would create a foam.

51. Cartridge according to any of preceding claim 49 or 50, wherein the foaming chamber comprises a rotatable foaming element arranged in the foaming chamber for performing a foaming process to produce the microfoam from a liquid foaming composition when driven rotatably and/or for performing a foam maintaining process to maintain a created foam when driven rotatably.

52. Cartridge according to preceding claim 50, wherein the foaming chamber comprises a positioning member on the bottom of the foaming chamber that holds the foaming element to be rotatable around a predefined axis during the foaming process,

53. Cartridge according to preceding claim 50, wherein the foaming chamber comprises a first outlet channel, namely a foam extraction opening, provided adjacent to or at the bottom part of the foaming chamber that is in fluid communication with the said foaming chamber for removing the microfoam from the foaming chamber.

54. Cartridge according to any of preceding claims 49 to 53, wherein the reaction unit defining the foaming chamber further has one or more of the following characteristics, and preferably all of the following characteristics:
a. a first inlet channel, namely a gas supply opening, that is in fluidical communication with the said foaming chamber for supplying a gas or gas mixture to the foaming chamber, whereby the first inlet channel is preferably provided at the top part of the foaming chamber,
b1. a second inlet channel, namely a foaming agent supply opening and/or a diluent supply opening or a combined foaming agent/diluent supply opening, that is in fluid communication with the said foaming chamber for supplying a mixture comprising a foaming agent and an aqueous diluent to the foaming chamber, whereby the second inlet channel is preferably provided adjacent to or at the bottom part of the foaming chamber, or
b2. a second inlet channel, namely a foaming agent supply opening and/or a diluent supply opening or a combined foaming agent/diluent supply opening, that is in fluid communication with the said foaming chamber for supplying an aqueous diluent to the foaming chamber, whereby the second inlet channel is preferably provided at the top part of the foaming chamber, provided together with a third inlet channel, namely a foaming agent supply opening, that is in fluid communication with the said foaming chamber for supplying a foaming agent to the foaming chamber, whereby the third inlet channel is preferably provided adjacent to or at the bottom part of the foaming chamber.

55. Cartridge according to any of preceding claims 49 to 54, wherein the reaction unit defining the foaming chamber further has a second outlet channel, namely a waste outlet opening, that is in fluid communication with the said foaming chamber for removing waste material from the foaming chamber whereby the second outlet channel is preferably provided adjacent to or at the bottom part of the foaming chamber.

56. Cartridge according to any of preceding claims 49 to 55, wherein the reaction unit body defining the foaming chamber further has a deflection element which comprises a surface which reduces a free axial-parallel height within the foaming chamber along a circumferential path, so that if a fluid, especially a foam, is rotated within the foaming chamber along a circumferential path, part of the foam is subjected to a forced deflection generating a downward fluid motion during the foaming process in direction to the rotating foaming element at the bottom part.

57. Cartridge according to any of preceding claims 49 to 56, wherein the reaction unit body is a sealed reaction unit body and/or a sterile or sterilizable reaction unit body.

58. Cartridge according to any of preceding claims 49 to 57, wherein the rotatable foaming element is configured as a disc with mixing element protruding radially and located around an outer perimeter of said disc, whereas the mixing features comprise one or more of the following: blades, rings, teeth, fins, rips, wings, arms, fingers, perforated plates, grid structures, or a ring-shaped helical spring.

59. Cartridge according to any of preceding claims 49 to 58, wherein the rotatable foaming element, being preferably a disc, is configured to be magnetically driven by a magnetic stirrer located outside the foaming chamber and preferably beneath the bottom part of the foaming chamber, and comprises a magnetically couplable element.

60. Cartridge according to any of preceding claims 49 to 59, wherein the rotatable foaming element has a centrally located bore, which is preferably open at one end for receiving the positioning member.

61. Cartridge according to any of preceding claims 49 to 60, wherein the positioning member is a centrally or eccentrically located shaft on the bottom of the foaming chamber and preferably has a cylindrical side wall with a rounded upper corner.

62. Cartridge according to any of preceding claims 49 to 61, wherein the reaction vessel further comprises a restriction member for restricting an upward movement of the rotatable foaming element, whereby said restriction member is preferably a rod-shaped element extending from the top part of the foaming chamber.

63. Cartridge according to any of preceding claims 49 to 62, wherein the deflection element is a deflector plate or deflector body located in the foaming chamber and/or a propellor provided on the rotating member.

64. Cartridge according to preceding claim 63, wherein the deflector body is a circular ring segment of trapezoidal basic structure with a beveled side towards the bottom of the foaming chamber and a bottom horizontal side situated above the rotatable foaming element to provide a gap between the deflector body and the upper margin of the rotatable foaming element, whereby the circular ring segment is preferably attached to the cylindrical wall of the foaming chamber, having more preferably a radial ring thickness which covers the mixing features of the rotatable disc.

65. Cartridge according to preceding claim 64, wherein the channel is situated under the bottom horizontal side of the deflector body.

66. Cartridge according to any of preceding claims 49 to 65, wherein the reaction unit body contains a gas distributor system, preferably selected from:
a deviation plate or body located in the foaming chamber beneath the first inlet channel to induce a lateral deflection of the gas stream entering the foaming chamber, or
a semispherical-, conical-, frustoconical- or dome-shaped top part of the foaming chamber with the first inlet channel at the top enabling a gas flow which is uniformly distributed over the horizontal cross-section of the foaming chamber.

67. Cartridge according to any of preceding claims 49 to 66, wherein the surface of an upper section of the foaming chamber is coated with a hydrophobic or superhydrophobic agent or is a morphologically structured (super) hydrophobic surface such as a superamphiphobic, a slippery liquid-infused porous surface (SLIPS), or a liquid-impregnated surface (LIS).

68. Cartridge according to any of preceding claims 49 to 60, wherein the reaction unit further comprises
a waste container defining a waste chamber containing an inlet for receiving the waste material from the foaming chamber,
a fluid conduit coupling the second outlet channel of the foaming chamber through a valve with the said inlet of the waste container, and
the waste container preferably comprising an outlet for pressure release.

69. Cartridge according to preceding claim 68, wherein the inlet is positioned in the top part of the waste container, preferably as the upper aperture of a hollow shaft positioned preferably in the center of the waste chamber.

70. Cartridge according to any of preceding claim 68 or 69, wherein the valve for controlling the flow of the waste material into the waste chamber is located on the outer surface or accessible from an outer surface of the cartridge and can be controlled by exerting an external pressure on said valve.

71. Cartridge according to any of preceding claims 68 to 70, wherein the inner surface of the waste chamber is coated with a defoaming agent or has a morphologically structured defoaming inner surface such as an anti-foaming surface such as a superamphiphobic, a slippery liquid-infused porous surface (SLIPS), or a liquid-impregnated surface (LIS).

72. Cartridge according to any of preceding claims 68 to 71, wherein a waste chamber volume is larger than a foaming chamber volume, preferably the waste chamber volume being from >=1 to <=3 times the foaming chamber volume.

73. Cartridge according to any of preceding claims 68 to 72, wherein the waste chamber and the foaming chamber are adjacent to each other, having a joint wall.

74. Cartridge according to any of preceding claims 68 to 73, wherein a fluidic connection between the foaming chamber and the waste chamber connects to the foaming chamber at a bottom of the foaming chamber and open into the waste chamber at a top region of the waste chamber.

75. Cartridge according to any of the preceding claims, wherein the cartridge further comprises, on its exterior, a fully closed surface, comprising, preferably consisting of, the housing body and multiple sealing members.

76. Cartridge according to preceding claim 75, wherein the sealing members are detachable glued tapes.

77. Cartridge according to any of the preceding claims, wherein the cartridge comprises, preferably within the hollow interior, an electronically readable identifier means, preferably selected from an RFID tag, a barcode, a data matrix code, a QR code or a different optical code.

78. An automated device for generating a therapeutical microfoam comprising a cartridge holder adapted to hold a replaceable cartridge according to any of the preceding claims.

79. The automated device according to preceding claim 78, wherein the device comprises a magnetic stirrer actor situated beneath the cartridge holder, for generating a rotating magnetic field for controlling the rotation of the foaming element around the positioning member upon inserting of a cartridge.

80. The automated device according to any of preceding claim 78 or 79, wherein the cartridge holder contains a gas feed port that is configured to fluidly engage with the gas supply port of a cartridge upon insertion in the holder, thereby providing a gas-tight seal of both ports, wherein the gas feed port or the system preferably contains a safety valve allowing a gas supply only for a cartridge being properly locked in the cartridge holder.

81. The automated device according to any of preceding claims 78 to 80, wherein the cartridge holder contains a diluent feed port that is configured to fluidly engage with the diluent supply port of the cartridge in the holder, thereby providing a fluid-tight seal of both ports, wherein the diluent feed port or the system preferably contains a safety valve allowing a diluent supply only for a cartridge being properly locked in the cartridge holder.

82. The automated device according to any of preceding claims 78 to 81, wherein the automated device contains a sensor that is configured to detect within the cartridge the presence or flow of a liquid in the fluid conduit coupling the diluent port and the second inlet channel of the foaming chamber, whereby said sensor is preferably a capacitive sensor or an ultrasonic sensor.

83. The automated device according to any of preceding claims 78 to 82, wherein the cartridge holder contains one or more guiding elements for restricting an insertion of the cartridge within the cartridge holder to a zero tolerance seat, wherein the guiding device preferably contains an, preferably at least two, retractable members arranged at the bottom of the cartridge holder and mate with respective orifices of the cartridge housing.

84. The automated device according to any of preceding claims 78 to 83, wherein the cartridge holder contains a fastening mechanism configured to fasten the cartridge to the cartridge holder, whereby the fastening mechanism preferably contains one or more members that are arranged in the lateral wall of the cartridge holder and can be extended to engage with respective features of the cartridge housing.

85. The automated device according to any of preceding claims 78 to 84, wherein the automated device contains a gas supply system containing:
at least one gas port for connection to at least one gas container, being preferably a gas bottle,
at least one gas conduit connecting the at least one gas port to the gas supply port via a valve for regulating the gas flow,
optionally a pressure compensation device for compensating for pressure transients in the pressure within the foaming chamber.

86. The automated device according to any of preceding claims 78 to 85, wherein the automated device contains a gas supply system containing
two gas ports for connection to two gas bottles containing different gases, preferably installed in the automated device and being more preferably an O₂ gas bottle and a CO₂ gas bottle,
a gas channel network between the two gas ports and the gas feed port,
a gas mixing device for mixing the two different gases in a selectable ratio, and
optionally a pressure compensation device for compensating for pressure transients in the pressure within the foaming chamber.

87. The automated device according to any of preceding claims 78 to 86, wherein the automated device contains a diluent supply system containing
a diluent port for connection to a diluent container, preferably installed in the automated device, and
a liquid conduit connecting the diluent port to the diluent feed port via a pump capable of transporting the diluent from the diluent container to the diluent feed port.

88. The automated device according to any of preceding claims 78 to 87, wherein the automated device contains a syringe-associated delivery system containing a drive unit moving an agitator which can perform at least one of the following actions:
detecting the correct connection of the inserted syringe to the foaming agent reservoir port of the said holding member,
detecting the complete filling of the syringe via the foaming agent reservoir port by detecting the final position of the plunger,
delivering a determined amount of foaming agent to the foaming chamber by pushing the plunger of the foaming agent-containing syringe to a controlled extent,
optionally detecting malfunctions of the syringe by determining the amount of pressure to be executed on the plunger of the syringe.

89. The automated device according to any of preceding claims 78 to 88, wherein the system further comprises:
a control computing unit, in which a control program is located connected with a user interface for controlling the foaming process, particularly by configuring at least one parameter of the foaming process and/or by initiating the foaming process, the at least one parameter being directly or indirectly selected from the following:
a gas concentration,
a mixing ratio of the gas mixture,
a concentration of the foaming agent in the mixture of foaming agent and diluent,
the total amount of the mixture of foaming agent and diluent, selection of the liquid composition,
the rotation speed and time of the rotatable foaming element,
the removal of waste material from the foaming chamber,
the cleansing of the foaming chamber as preparation for a further foaming process,
the removal of the cartridge from the cartridge holder,
wherein the automated device is configured to perform the foaming process based on the configured parameter automatically, and particularly the mixing of the gases according to the configured mixing ratio of the gas mixture automatically.

90. The automated device according to any of preceding claims 78 to 89, wherein there is a conjunction of two gas supply conduits external of the cartridge, so as to unite a gas supply of two gas sources located within the device into one common fluidical stream into the cartridge.

91. The automated device according to any of preceding claims 78 to 90, wherein the cartridge holder comprises a replaceably inserted and connected cartridge according to any of preceding claims 1 to 77.

92. The automated device according to preceding claim 91, wherein the device is electrically powered.

93. The automated device according to any of preceding claims 78 to 92, wherein a computer program comprises instructions for performing steps which, when the program is executed by a computer, cause the device to carry out the following steps:
a. Providing a graphical user interface (GUI) for controlling a foaming process for producing a microfoam, particularly by configuring at least one parameter of the foaming process and/or by initiating the foaming process, the at least one parameter being selected from: a gas concentration, a mixing ratio of the gas mixture, a selection of the liquid composition, a mixing ratio of the agent solution, a concentration of the agent solution in the microfoam, a foaming profile (204);
b. providing at least one graphical element (201) as part of the user interface (200) for selecting or configuring the at least one parameter;
c. providing at least one graphical control element (202) for directing the computer program, in particular a graphical control element (202) for cancelling the computer program and/or a graphical control element (202) for continuing the computer program and/or a graphical control element (202) for moving one step backwards in the computer program and/or a graphical control element for rinsing the foaming chamber and producing another amount of foam without exchanging the cartridge;
d. providing at least one graphical section (203) as part of the user interface (200) showing at least one instructional image (205) and/or at least one informational image (206) for guiding or informing a user.

94. The automated device according to preceding claim 93, wherein the foaming profile (204) comprises at least one of the following parameters:
profile name and description,
names of foaming agent, diluent, gasses,
list of compatible cartridges,
available selection of agent input concentration,
valid range of agent input concentration,
volume of agent to be injected into the cartridge,
available selection of agent concentration in foam,
available selection of gas ratios,
detailed instructions of how to prepare the microfoam,
limit of keeping the microfoam fresh after preparation,
time limit for use of cartridge.

95. The automated device according to any of preceding claims 78 to 94, wherein a timer defines a maximum time for using a prepared microfoam, signaling when the predefined maximum time has been used up.

96. A method for automatically producing a therapeutic microfoam comprising the following steps:
a. providing an automated device according to any of preceding claims 78 to 95;
b. inserting a cartridge according to any of preceding claims 1 to 77 into the cartridge holder of the automated device;
c. selecting a foaming agent concentration using a user interface,
d. preferably selecting a ratio for the mixture of two gases using a user interface,
e. providing, preferably injecting, a foaming agent, preferably through a sealing member, of a foaming agent supply port; or providing within the sealed cartridge a foaming agent;
f. starting a foam generation process using a user interface; until a suitable microfoam has been obtained in the reaction unit,
g. once the foam has been obtained, extracting microfoam through a foam extraction port of the cartridge.

97. The method according to preceding claim 96, with the additional steps of:
optionally flushing the foaming chamber with a selected gas or gas mixture or rinsing with the aqueous diluent, being preferably saline,
introducing the selected gas or gas mixture into the foaming chamber via the first inlet channel,
introducing a mixture of the foaming agent and the aqueous diluent into the foaming chamber via the second inlet channel, or alternatively, introducing the aqueous diluent into the foaming chamber via the second inlet channel and the foaming agent into the foaming chamber via the third inlet channel.

98. The method according to any of preceding claim 96 or 97, with the additional step of
initiating a rinsing process in the foaming chamber by using a user interface.

99. The method according to any of preceding claims 96 to 98, with the additional step of
repeating the process according steps f and g, preferably e, f and g, or d, e, f and g, thereby maintaining the same cartridge; or
exchanging the cartridge by removing the used cartridge and inserting a new, sealed cartridge.

100. A therapeutic microfoam,
preferably obtained by a method according to any of preceding claims 96 to 99 and/or using a cartridge according to any of preceding claims 1 to 77 and/or using a device according to any of preceding claims 78 to 95,
with one or more of the following properties:
the microfoam has a nitrogen content of less than 2%, preferably of less than 1% and more preferably of less than 0.8%; and/or
the microfoam has a number of geometrical impurities being less than 30% of the bubble count, preferably less than impurities being less than 10% of the bubble count and more preferably less than 1% of the bubble count; and/or
the microfoam has a mean bubble size of less than 100 μm, preferably less than 80 μm, and more preferably less than 50 μm, measured 45 seconds after foam preparation; and/or
the microfoam has a Sauter mean radius of less of less than 200 μm, preferably less than 150 μm, and more preferably less than 100 μm, measured 45 seconds after foam preparation; and/or
the microfoam has a moisture content of less of less than 20% (w/w), being preferably between 8 and 12% (w/w).

101. Use of the microfoam
according to preceding claim 100 and/or of a microfoam obtained by a method according to any of preceding claims 96 to 99 and/or using a cartridge according to any of preceding claims 1 to 77 and/or using a device according to any of preceding claims 78 to 95,
for the therapeutic treatment of a human patient's (i) varicose veins of CEAP classifications C1, C2A, C2S, C3, C4, C5 or C6 or (ii) haemorrhoids of Grade I, Grade II, Grade III or Grade IV, or (iii) venous malformations, or (iv) varicocele, or (v) pelvic varicose veins, or (vi) vulvar varicose veins, or (vii) hepatocarcinoma, or (viii) esophageal varices, by injecting a sclerosant solution, in the form of the microfoam, into an affected vessel, thereby displacing blood in the vessel and leading to a closure of the affected vessel.
